(19) United States
(12) Patent Application Publication
(10) Pub. No.: US 2016/0045498 A1
(43) Pub. Date: Feb. 18, 2016

(54) METHODS OF TREATING DISEASES CHARACTERIZED BY EXCESSIVE WNT SIGNALLING

(71) Applicant: THE WALTER AND ELIZA HALL INSTITUTE OF MEDICAL RESEARCH, Melbourne, Victoria (AU)

(72) Inventors: Matthias Robert Walter Ernst, Melbourne (AU); Toby James Phesse, Melbourne (AU); Michael Buchert, Melbourne (AU); Emma Charlotte Stuart, Melbourne (AU)

(21) Appl. No.: 14/782,575

(22) PCT Filed: Apr. 4, 2014
(86) PCT No.: PCT/AU2014/000370

§ 371 Date: Oct. 5, 2015

(30) Foreign Application Priority Data
Apr. 4, 2013 (AU) 2013901161

(51) Int. Cl. A61K 31/506; A61K 31/5377

(52) U.S. Cl. CPC A61K 31/506; A61K 31/5377

(57) ABSTRACT

The present invention relates to a method of treating or preventing a disease characterized by excessive Wnt signalling, such as colorectal cancer, by administering a compound which inhibits the activity of one or more of JAK2, JAK1 or TYK2.

### FIELD OF THE INVENTION

The present invention relates to a method of treating or preventing a disease characterized by excessive Wnt signalling, such as colorectal cancer, by administering a compound which inhibits the activity of one or more of JAK2, JAK1 or TYK2.

### BACKGROUND OF THE INVENTION

The Wingless (Wnt) signalling pathway is involved in the early development of complex, multi-cellular organisms controlling early axis formation, limb patterning and organogenesis. Wnt ligands are secreted, palmitoylated glycoproteins which play a central role in embryogenesis and tissue homeostasis of adult organisms (Capdevila et al., 2001; Logan and Nusse, 2004). Abnormal Wnt signalling is often associated with severe human diseases such as cancer, osteoporosis and other degenerative diseases (Capdevila et al., 2001; Logan and Nusse, 2004; Polakis, 2000).

In the canonical Wnt pathway, the signal is transduced by FZD-LRP heterodimeric receptors, regulating stability and nuclear translocation of the transcriptional co-activator β-catenin. Some Wnts activate non-canonical, β-catenin-independent cascades such as the Wnt/Ca2+ and the Wnt/planar cell polarity (PCP) pathway. In addition, Wnt ligands can bind to receptor tyrosine kinases such as Ror and Ryk the latter playing a role in neuronal development. Hence, Wnt signalling describes a highly organised network of different ligands, receptors and downstream effectors controlling complex cellular responses (van Amerongen et al., 2009).

Most colon cancers arise when there are inactivation mutations in the APC gene which forms part of the Wnt signalling pathway. The normal APC protein prevents the accumulation of cytosolic and nuclear β-catenin by mediating its phosphorylation and degradation. The majority of mutations in the APC gene (both germline and somatic) lead to premature truncation of the APC protein and loss of its capacity to regulate β-catenin turnover. Loss of functional APC results in the nuclear accumulation of β-catenin and formation of a complex with Tcf-4 that transcriptionally activates Wnt target genes. The genetic events occurring after the APC gene mutation are dependent on the underlying genetic instability-chromosomal instability, germ line mutations in DNA mismatch repair enzymes, and CpG island hyper-methylation phenotype [CIMP+] (Noffsinger, 2009). Many other cancers (including some colorectal cancer) with aberrant canonical Wnt signaling, however, arise from constitutive activating mutations in β-catenin that render the protein resistant to degradation.

There is a need for further methods of treating diseases characterized by excessive Wnt signalling such as cancers in patients with, for example, an inactivated APC gene.

### SUMMARY OF THE INVENTION

The canonical Wnt signalling pathway is indispensable for intestinal maintenance during day-to-day tissue homeostasis of the intestinal lining, which completely renews every five to seven days in humans and mice. Moreover, there is also an absolute requirement for the canonical Wnt pathway to enable epithelial regeneration in response to DNA damage, where an initial wave of apoptosis denudes the intestine from its epithelium to trigger extensive proliferation of intestinal epithelial cells (IEC) thereafter (Ashton et al., 2010). Finally, aberrant canonical Wnt signalling is required for the formation of intestinal tumors, including those that give rise to colorectal cancer. However, epithelial Stat3 also promotes survival and proliferation of normal and mutated IEC in a mouse model of colitis-associated colorectal cancer (Bollrath et al., 2009). Accordingly, excessive Stat3 activity protects against experimentally induced colitis in gp130^{F/F} mice, where the gp130 mutation prevents Socs3 binding and associated suppression of signalling by IL-6 family cytokines. Surprisingly, the inventors have found that inhibiting the gp130/Stat3 pathway through the activity of one or more gp130-associated Jak tyrosine kinases (i.e. JAK2, JAK1 or TYK2) is useful in the treatment or prevention of diseases characterized by excessive canonical Wnt signalling. Importantly, the latter occurs without affecting signalling of the canonical Wnt pathway and therefore does not negatively impact on normal epithelial homeostasis.

Thus, in a first aspect the present invention provides a method of treating or preventing a disease characterized by excessive Wnt signalling in a subject, the method comprising administering to the subject a compound which inhibits the activity of one or more of JAK2, JAK1 or TYK2.

In another aspect, the present invention provides for the use of a compound which inhibits the activity of one or more of JAK2, JAK1 or TYK2 for the manufacture of a medicament for treating or preventing a disease characterized by excessive Wnt signalling in a subject.

In a further aspect, the present invention provides for the use of a compound which inhibits the activity of one or more of JAK2, JAK1 or TYK2 as a medicament for treating or preventing a disease characterized by excessive Wnt signalling in a subject.

In an embodiment, the disease is cancer or a bone related disorder. Examples of cancers which can be treated by methods of the invention include, but are not limited to, colorectal cancer, hepatocellular cancer, medullablastoma, ovarian cancer, pancreatic cancer, gastric cancer, endometrial cancer, adrenocortical cancer, pituitary gland cancer, biliary tract cancer, kidney cancer, soft tissue cancer, intestinal cancer, breast cancer, oesophageal cancer, gliobalstoma, lung cancer, prostate cancer and thyroid cancer. In a preferred embodiment, the cancer is colorectal cancer such as sporadic colon cancer or familial adenomatous polyposis syndrome (FAP). In another preferred embodiment, the colorectal cancer is FAP. In yet a further preferred embodiment, the subject does not have an inflammatory disorder such as colitis or Crohn's disease.

In an embodiment, the bone related disorder is osteoporosis.

In an embodiment, the subject has one or more mutations or epigenetic modifications in a gene encoding a protein which results in excessive Wnt signalling, and wherein the gene can be, but is not limited to, APC, TCF7L2 (=TCF4), CTNNB1, WTX, AXIN1, DKK or a SFRP. In a preferred embodiment, the subject has one or more mutations or epigenetic modifications in the APC gene.

In an embodiment, the method further comprises testing the subject to determine whether they have a disease characterized by excessive Wnt signalling before administering the compound. For example, if it is determined that a subject has cancer, the subject could be screened to establish whether the cancer is characterized by excessive Wnt signalling by testing the subject or a sample therefrom (for example a sample obtained from the subject comprising genomic DNA) for the presence of one or more mutations or epigenetic modifications in a gene encoding a protein involved in Wnt signalling such as, but not limited to, APC, TCF7L2 (=TCF4), CTNNB1, WTX, AXIN1, DKK or a SFRP.

In a preferred embodiment, the subject is a human.

In an embodiment, the compound is selected from Ruxolitinib, Baricitinib, Lestaurtinib, Pacritinib, CEP-33779, SB1578, SB1317, TG101348, TG101209, CYT387, AZD1480, AZ960, LY2784544, BMS911543, SGI-1252, MK0457, XL019, AG490, AT9283, NVP-BSK805, AC430 and GLPG0634. In a further embodiment, the compound is AZD1480.

In an embodiment, the compound is a selective inhibitor of JAK1. Examples of selective JAK1 inhibitors include, but are not limited to, GLPG0634,

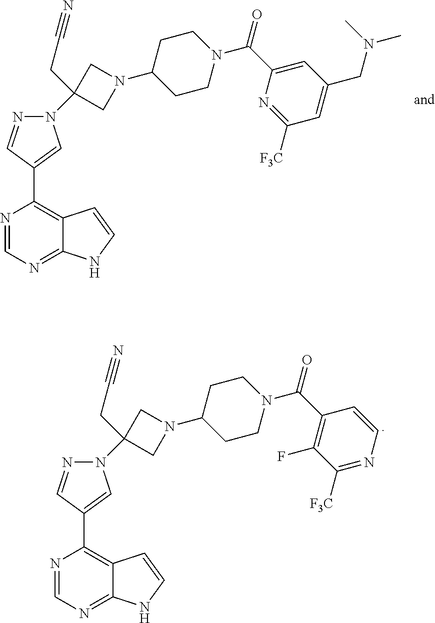 [SMILES: CN(C)Cc1cc(C(=O)N2CCC(N3CC(CC#N)(n4cc(-c5ncnc6[nH]ccc56)cn4)C3)CC2)nc(C(F)(F)F)c1 ; N#CCC1(n2cc(-c3ncnc4[nH]ccc34)cn2)CN(C2CCN(C(=O)c3ccnc(C(F)(F)F)c3F)CC2)C1]

In an alternate embodiment, the compound is a selective inhibitor of JAK2. Examples of selective JAK2 inhibitors include, but are not limited to, Pacritinib, CEP-33779, SB1578, TG101348, TG101209, AZD1480, AZ960, LY2784544, BMS911543, SGI-1252, MK0457, XL019 and NVP-BSK805. In a further embodiment, the compound is AZD1480.

In a further alternate embodiment, the compound is a selective inhibitor of JAK1 and JAK2. Examples of selective JAK1 and JAK2 inhibitors include, but are not limited to, Ruxolitinib, Baricitinib and CYT387.

In yet a further alternate embodiment, the compound is a selective inhibitor of TYK2. Examples of selective TYK2 inhibitors include, but are not limited to, TG101348,

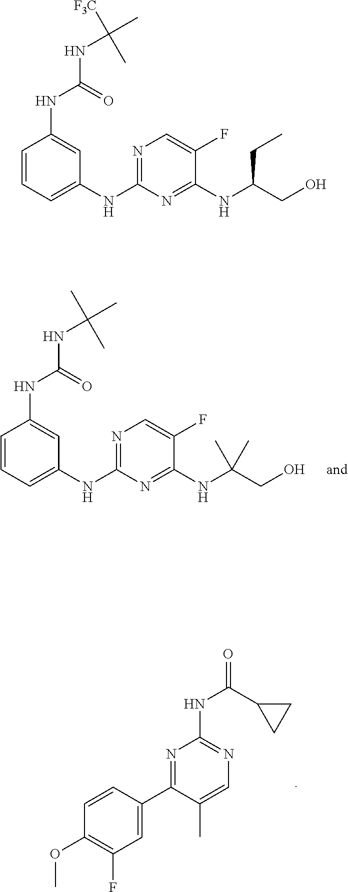 [SMILES: CC[C@@H](CO)Nc1nc(Nc2cccc(NC(=O)NC(C)(C)C(F)(F)F)c2)ncc1F ; CC(C)(C)NC(=O)Nc1cccc(Nc2ncc(F)c(NC(C)(C)CO)n2)c1 ; COc1ccc(-c2nc(NC(=O)C3CC3)ncc2C)cc1F]

In another embodiment, the compound is an antibody which binds one or more of JAK2, JAK1 or TYK2.

In yet a further embodiment, the compound reduces transcription and/or translation of a gene encoding one or more of JAK2, JAK1 or TYK2. In a preferred embodiment, the compound which reduces transcription and/or translation of a gene encoding one or more of JAK2, JAK1 or TYK2 is a polynucleotide. Examples of such polynucleotides include, but are not limited to, an antisense polynucleotide, a sense polynucleotide, a catalytic polynucleotide, a microRNA and a double stranded RNA.

The present invention also allows for patients to be selected who are more likely to respond to treatment with a compound which inhibits the activity of one or more of JAK2, JAK1 or TYK2. As the skilled person will appreciate, this avoids the inconvenience and cost associated with administering the compound to patients who are unlikely to respond and where a different treatment is likely to be more suitable. Thus, in another aspect the present invention provides a method of treating or preventing cancer in a subject, the method comprising;

i) analysing a sample from the subject to determine whether cells of the subject have excessive Wnt signalling, and

ii) if excessive Wnt signalling is detected in step i), administering to the subject a compound which inhibits the activity of one or more of JAK2, JAK1 or TYK2.

In an embodiment, the subject has cancer and the sample comprises cancerous cells.

In an embodiment, step i) comprises analysing a nucleic acid, such as DNA, in the sample.

In an embodiment, step i) comprises analysing the sample for one or more mutations or epigenetic modifications in a gene encoding a protein involved in Wnt signalling, and wherein the gene is selected from APC, TCF7L2 (=TCF4), CTNNB1, WTX, AXIN1, DKK or a SFRP.

Also provided is the use of a compound which inhibits the activity of one or more of JAK2, JAK1 or TYK2 for the manufacture of a medicament for treating or preventing a disease characterized by excessive Wnt signalling in a subject, wherein the subject has been identified as having excessive Wnt signalling.

Furthermore, provided is the use of a compound which inhibits the activity of one or more of JAK2, JAK1 or TYK2 as a medicament for treating or preventing a disease characterized by excessive Wnt signalling in a subject which has been identified as having excessive Wnt signalling.

The present invention also enables more informative clinical trials to be conducted using a compound which inhibits the activity of one or more of JAK2, JAK1 or TYK2. More specifically, the efficacy of the compound can more appropriately be assessed when all individuals in the trial have excessive Wnt signalling. Accordingly, in a further aspect, the present invention provides a method of stratifying individuals in a clinical trial of a compound which inhibits the activity of one or more of JAK2, JAK1 or TYK2, the method comprising,

i) analysing samples from the individuals to determine whether cells in the samples have excessive Wnt signalling, and,

ii) selecting individuals for the trial who have excessive Wnt signalling.

In an embodiment, the individuals have cancer and the samples comprise cancerous cells.

In an embodiment, step i) comprises analysing a nucleic acid, such as DNA, in the samples.

In an embodiment, step i) comprises analysing the samples for one or more mutations or epigenetic modifications in a gene encoding a protein involved in Wnt signalling, and wherein the gene is selected from APC, TCF7L2 (=TCF4), CTNNB1, WTX, AXIN1, DKK or a SFRP.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying FIGS.

### BRIEF DESCRIPTION OF THE ACCOMPANYING DRAWINGS

FIG. 1. Signalling through gp130/Stat3 regulates intestinal regeneration.

a Immunohistochemical analysis for the proliferation marker PCNA on sections of small intestines of wild-type (Wt), gp130^{F/F} (F/F), Stat3^{+/-}, gp130^{F/F};Stat3^{+/-} (F/F Stat3^{+/-}), gp130^{ΔStat/ΔStat} (Δ/Δ) and gp130^{ΔStat/+} (Δ/+) as well as of Wt and F/F mice treated with Jak2 kinase inhibitor AZD1480 (Jak2i; 30 mg/kg) 72 h after exposure to γ-irradiation. Scale bar=100 μm.

b Enumeration of intact crypts in cross sections from the proximal small intestine stained for PCNA obtained 72 h after exposure of mice of the indicated genotype to γ-irradiation. (*p≦0.04; n=3 mice).

c Organoids cultured from small intestines collected from wild-type (Wt) and gp130^{F/F} (F/F) mice, as well as organoids from Wt mice that were cultured in the presence of the Stat3 inhibitor S3I-201 (Stat3i, 7.5 μM) from day 3 after seeding. Arrows point to budding crypt-like structures. Scale bar=50 μm.

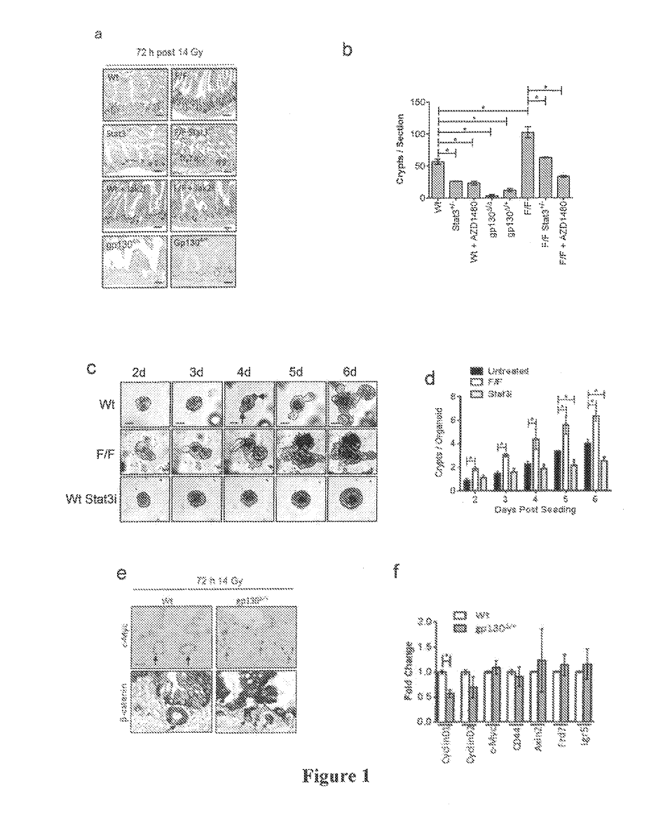

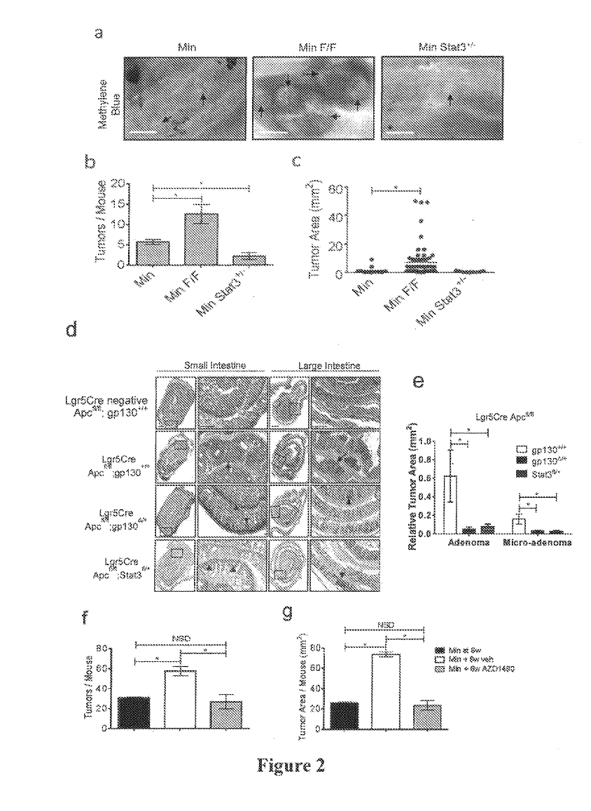

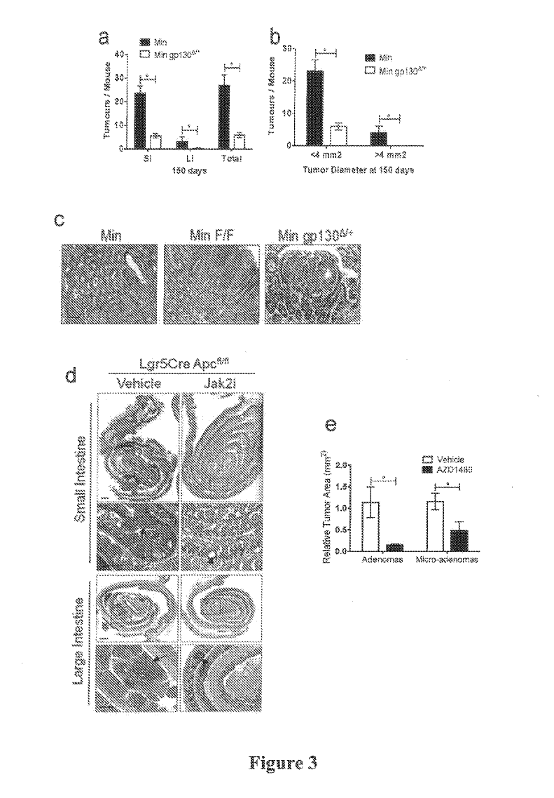

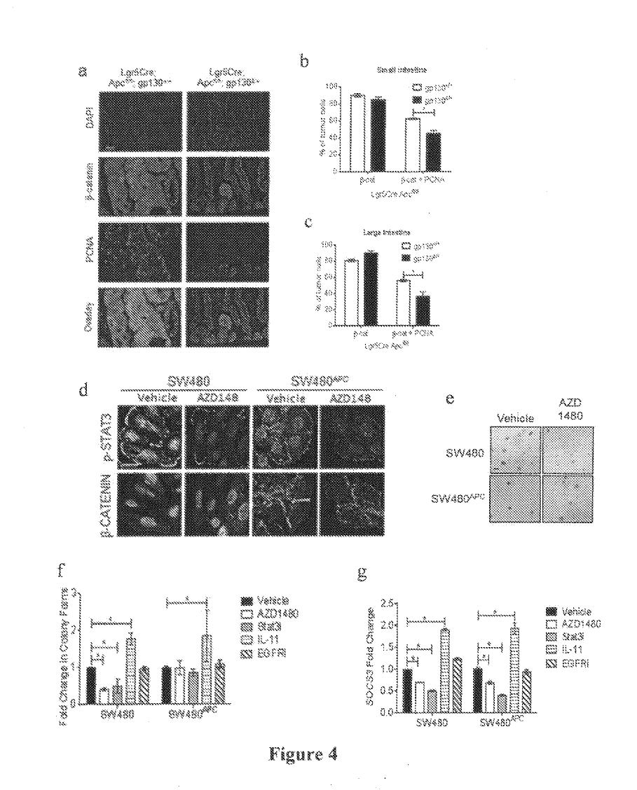

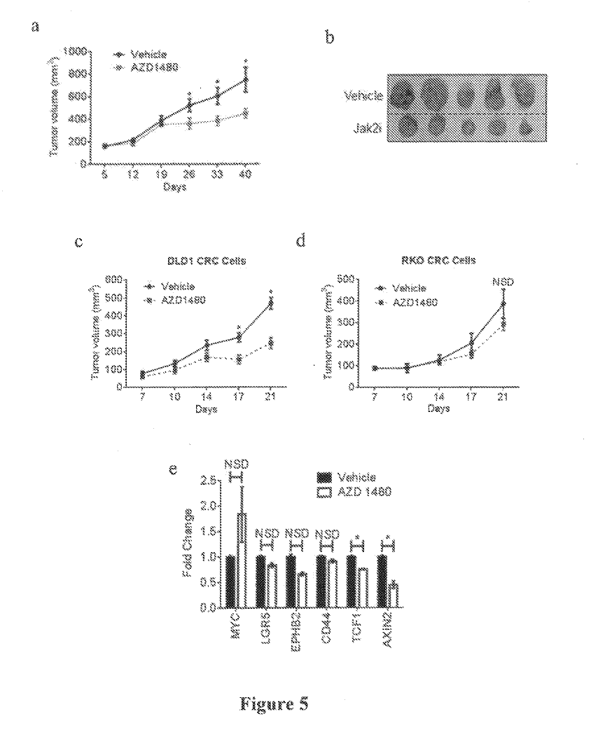

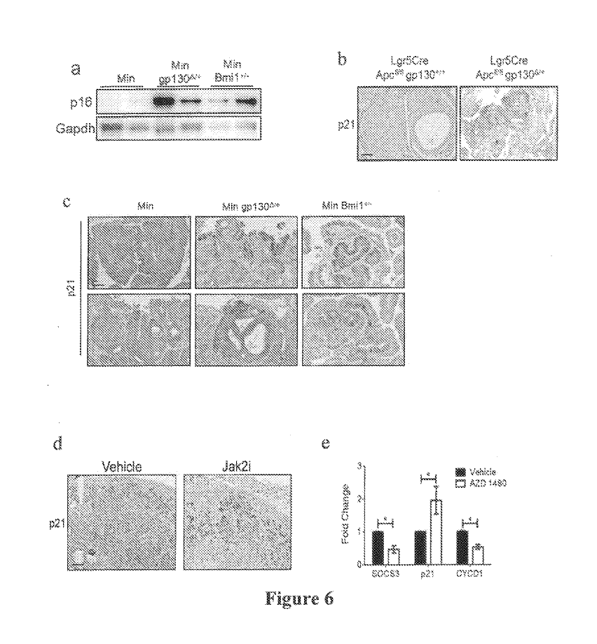

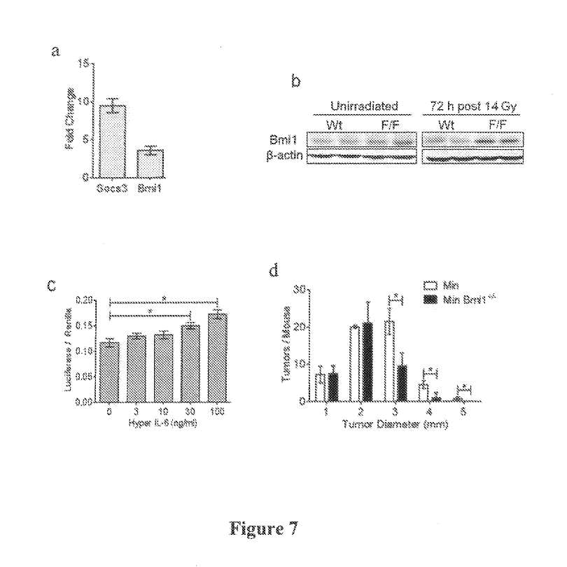

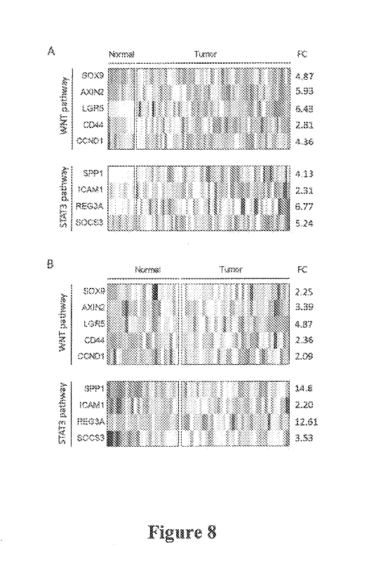

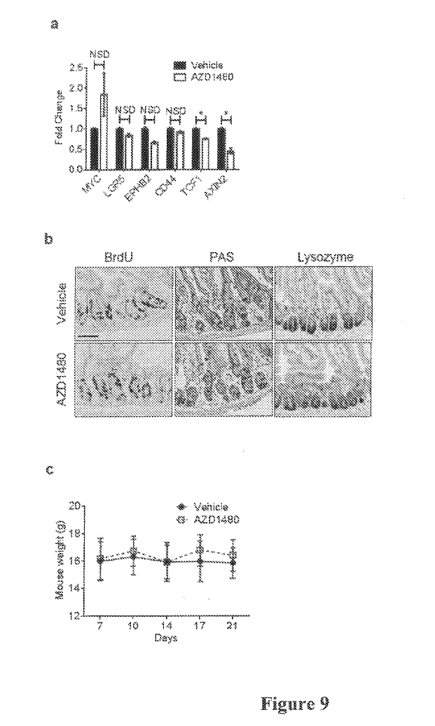

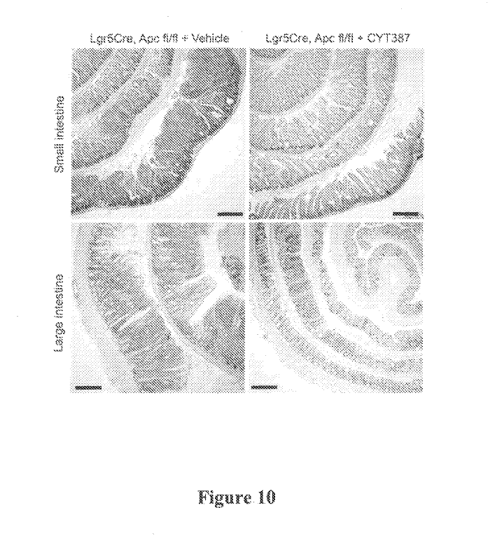

d Enumeration of crypts on individual organoids from experiment illustrated in FIG. 1c. (*p≦0.018; Two-sided t-test on triplicate cultures and repeated).

e Immunohistochemical analysis for c-Myc and β-catenin expression on sections of small intestines from wild-type (Wt) and gp130^{ΔStat/+} mice 72 h after γ-irradiation. Staining for both markers occurs in regenerating crypt (black arrows) and non-regenerating epithelium (red arrows). Scale bar=50 μm.

f RT-qPCR profiling of β-catenin target gene expression in IECs isolated from small intestines of Wt and gp130^{ΔStat/+} mice 72 h after γ-irradiation.

FIG. 2. Signalling through gp130/Jak2/Stat3 regulates mutant Apc-mediated intestinal tumourigenesis.

s. Methylene blue stain to visualize emerging colonic tumours in 100 day old Apc^{Min/+} (Min), Apc^{Min/+};gp130^{F/F} (Min F/F) and Apc^{Min/+};Stat3^{+/-} (Min Stat3^{+/-}) mice on a 129SvJ×C57Bl/6J mixed genetic background. Scale bar=500 μm.

b, c Enumeration of total number and area of intestinal tumours in individual 100 day old mice of the indicated genotypes (*p≦0.02; n=5 mice).

d Tumour burden visualized on H&E stained sections of small and large intestines collected 5 weeks after tamoxifen-induced conditional Apc deletion in adult Lgr5^{CreERT2}-negative Apc^{fl/fl};gp130^{+/+} mice or Lgr5^{CreERT2}- positive Apc^{fl/fl} or Apc^{fl/fl}; gp130^{ΔStat/+} (Δ/+) or Apc^{fl/fl}; Stat3^{flox/+} mice. Higher magnifications of the boxed areas are provided and arrowheads point to neoplastic foci whilst arrows point to tubular adenomas. Scale bars=1 mm.

e Quantification of intestinal tumour burden of mice using the Metamorph analysis tool (Barker et al., 2009) after tamoxifen-induced conditional Apc deletion in adult Lgr5^{CreERT}2;Apc^{fl/fl} mice that also received daily administration of AZD1480 (30 mg/kg) or vehicle during this time. (*p≦0.04; n=3 mice).

f, g Enumeration of total number and area of intestinal tumours in individual 6 week old Apc^{Min} mice (black bars) and 12 week old Apc^{Min} mice having been treated daily with AZD1480 (Jak2i; 30 mg/kg)(grey bars) or vehicle (white bars) for the last 6 weeks (NSD=not significantly different. *p≦0.04; n=3 mice).

FIG. 3. Gp130/Jak2/Stat3 signalling limits mutant Apc-driven intestinal tumourigenesis.

a , b Enumeration of tumour burden and tumour size in the small intestines (SI) and large intestines (LI) of 150 day old Apc^{Min/+} (Min) and Apc^{Min/+};gp130^{ΔStat/+} mice (Min gp130^{Δ/+}) (*p≦0.002; n=8 mice).

c Intestinal tumours of 100 day old Apc^{Min/+} (Min), Apc^{Min/+};gp130^{F/F} (Min F/F) and Apc^{Min/+};gp130^{ΔStat/+} (Min gp130^{Δ/+}) are non invasive and display characteristics of tubular adenoma. Scale bar=100 μm.

d Intestinal tumour burden 5 weeks after tamoxifen-induced conditional Apc deletion in adult Lgr5^{CreERT2};Apc^{fl/fl} mice that also received daily administration of AZD1480 (Jak2i; 30 mg/kg) or vehicle during this time. Higher magnifications of the boxed areas are provided and arrows point to tubular adenomas whilst arrowheads point to micro-adenomas. Scale bars=1 mm.

e Quantification of intestinal tumour burden of mice using the Metamorph analysis tool (Barker et al., 2009) after tamoxifen-induced conditional Apc deletion in adult Lgr5^{CreERT2};Apc^{fl/fl} mice that also received daily administration of AZD1480 (30 mg/kg) or vehicle during this time. (*p≦0.04; n=3 mice).

FIG. 4. Wnt signalling is insufficient to promote tumour growth in vivo or induce colony formation of human CRC cells.

a Analysis of β-catenin and PCNA expression by co-immunofluorescence of intestinal tumour sections from adult Lgr5^{CreERT2};Apc^{fl/fl};gp130^{+/+} and Lgr5^{CreERT2};Apc^{fl/fl};gp130^{ΔStat/+} (Δ/+) mice 5 weeks after tamoxifen-induced conditional Apc deletion. Sections were counterstained with DAPI to visualize nuclei. Scale bar=50 μm.

b, c Quantification of epithelial cells in tumours from Lgr5^{CreERT2};Apc^{fl/fl};gp130^{+/+} and Lgr5^{CreERT2};Apc^{fl/fl}; gp130^{ΔStat/+} (Δ/+) mice from FIG. 4a simultaneously stained for β-catenin and PCNA. Numbers were normalized for the total number of tumour-associated IECs using the DAPI-stained nuclei as reference (*p≦0.04; n=3 mice).

d Immunofluorescence staining for β-CATENIN or tyrosine-705 phosphorylated STAT3 in an isogenic pair of SW480 cells grown in the presence of 1 μM AZD1480 (Jak2i) or vehicle. SW480 cells are APC mutant and lack expression of functional APC protein, while SW480^{APC} cells have been reconstituted to express wild-type APC. Scale bar=20 μm.

e Photograph of SW480 and SW480^{APC} CRC cells grown for 10 days under colony forming conditions in soft agar in the presence of vehicle or 1 μM of Jak2 inhibitor AZD1480 (Jak2i) (Scale bar=200 μm)

f Colony forming capacity of an isogenic pair of SW480 cells scored 10 days after the indicated treatment. (*p≦0.04; triplicate cultures and repeated).

g RT-qPCR expression analysis for the STAT3 target gene SOCS3 in SW480 and SW480^{APC} CRC cells grown for 10 days under colony forming conditions in soft agar in the presence of the indicated treatment. Expression in the vehicle-treated cultures was arbitrarily set as 1. (*p≦0.04; triplicate cultures in each of two repeats).

FIG. 5. Wnt signalling is insufficient to promote tumour growth in vivo or induce colony formation of human CRC cells.

a Tumour xenograft burden in BALB/c nude mice injected s.c. with SW480 cells in matrigel and receiving daily gavage with AZD1480 (30 mg/kg) or vehicle starting on the 5^{th} day after cell inoculation. (*p≦0.018; two sided t-test; n=12 mice),

b Photograph of xenograft tumours at time of harvest from experiment detailed above in (a).

c, d Tumour xenograft burden in BALB/c nude mice injected s.c. with DLD1 cells or RKO cells in PBS and receiving daily gavage with AZD1480 (30 mg/kg) or vehicle starting on the 7^{th} day after cell inoculation. (*p≦0.0004; two sided t-test; n=8).

e RT-qPCR for Wnt target genes from RNA extracted from SW480 xenografts in FIG. 3a. (*p≦0.04; triplicate cultures and repeated, NSD, not significantly different).

FIG. 6. Suppression of gp130/Jak2/Stat3 signalling induces Bmi-1 dependant growth arrest in Wnt-dependent intestinal tumours.

a Western blot analysis for p16 expression in intestinal tumours of 18 week old Apc^{Min/+} (Min), Apc^{Min/+};gp130^{ΔStat/+} (Min gp130^{Δ/+}) and Apc^{Min/+};Bmi1^{+/-} (Min Bmi1^{+/-}) mice. Gapdh was probed for loading control. Two different tumors are shown for each genotype.

b Immunohistochemical analysis for the cell cycle inhibitor p21 on sections of intestinal tumours from Lgr5^{CreERT2};gp130+/+ and Lgr5^{CreERT2};Apc^{fl/fl};gp130^{ΔStat/+} (Δ/+) mice 36 days following induction with tamoxifen showing an increase in p21 in Lgr5Cre⁺;Apc^{fl/fl};gp130^{ΔStat/+} (Δ/+) tumours. Scale bar=100 μm.

c Immunohistochemical analysis of p21 expression in intestinal tumours of 18 week old Apc^{Min/+} (Min), Apc^{Min/+}; gp130^{ΔStat/+} (Min gp130^{Δ/+}) and Apc^{Min/+};Bmi1^{+/-} (Min Bmi1^{+/-}) mice. Two different tumours are shown for each genotype. Scale bar=100 μm.

d Immunohistochemical analysis of p21 expression in xenograft tumours following 35 days of treatment with Jak2 inhibitor AZD1480 or vehicle. Scale bar=100 μm.

e RT-qPCR expression analysis for SOCS3, p21 and CYCLIN D1 in SW480 xenografts from FIG. 5a and b (p=0.04; n=3 mice).

FIG. 7. Suppression of gp130/Jak2/Stat3 signalling induces Bmi-1 dependant growth arrest in Wnt-dependent intestinal tumours.

a RT-qPCR expression analysis for Socs3 and Bmi1 in IECs isolated from gp130^{F/F} mice 30 minutes after an i.p. injection of 5 μg of recombinant IL-11 (p=0.04; n=3 mice).

b Western blot analysis of Bmi1 expression in IECs isolated from wild-type (Wt) and gp130^{F/F} (F/F) mice either unirradiated or 72 hours after γ-irradiation. Each lane represents a separate mouse and β-actin expression was used as a loading control.

c Luciferase activity in 293T cells conferred by co-transfection of a pBmi1(int1)-luc and pCMV-ren plasmids in response to 24 hour stimulation with the indicated concentrations of Hyper IL-6. Firefly luciferase activity was normalized to Renilla luciferase activity and expressed as Relative Luciferase Units) upon exposure to increasing doses of Hyper IL-6 (*p≦0.05; Student's t-test, triplicate cultures from twice repeated experiments).

d Enumeration of intestinal tumours classified by size in 18 week old Apc^{Min/+} and Apc^{Min/+};Bmi1^{+/-} mice (*p≦0.04; n=3 mice).

FIG. 8. mRNA microarray expression levels of bona fide WNT and STAT3 target genes from CRC and normal biopsies.

Oncomine (oncomine.org) derived data is comprised of 12 normal samples and 70 colon samples from the Hong et al. (2010) study (GSE9348) (A), and 24 normal samples and 36 tumor samples from the Skrzypczak et al. (2010) study (GSE20916) (B). Z-scores were calculated by subtracting the mean for the corresponding gene probe and then dividing by the standard deviation. Individual samples (vertically aligned) were ordered according to the average of the Z-scores. Mann-Whitney U tests were performed for each of the genes by comparing normal v tumor; p<0.001 was obtained for each gene analysed. FC, fold change.

FIG. 9. Homeostatic turnover of the intestinal epithelium in mice treated with AZD1480

a qRT-PCR expression analysis for Wnt target genes from RNA extracted from AZD1480 or vehicle-treated BALB/c-nude hosts used for the xenograft experiments (*p<0.04, n≧5 mice).

b Immunohistochemical analysis of cell proliferation (BrdU), goblet cells (Periodic Acid/Schiff, PAS) and Paneth cells (lysozyme) in the small intestines of BALB/c-nude mice treated for 14 days with AZD1480 (30 mg/kg, daily) or vehicle Control. Scale bar 50 μm.

c Weight of BALB/c-nude mice treated with either vehicle or AZD1480 (30 mg/kg, daily) between days 5 and 21 of xenograft experiment (n=8).

FIG. 10. Histological assessment of intestinal tumour burden following CYT387 administration for induction of aberrant Wnt activation

10 week old Lgr5CreERT2;Apcfl/fl mice received four i.p injections with tamoxifen (10 mg/ml; 300 ul, 200 ul, 200 ul, 200 ul) over four consecutive days. Three days after the last injection, mice received daily oral gavages with either vehicle (tap water, pH 2.0) or CYT387 (30 mg/kg) for four weeks. Regime was 5 days treatment followed by 2 days off treatment.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the phrase “disease characterized by excessive Wnt signalling” refers to conditions where the levels of Wnt signalling are abnormal through the canonical pathway. Typically, such diseases are the result of one or more mutations or epigenetic modifications in genes encoding proteins involved in Wnt signalling such as, but not limited to, APC (adenomatous polyposis coli), TCF7L2 (Transcription factor 7-like 2) (=TCF4), CTNNB1 (β-catenin), WTX (Wilms' tumor gene), AXIN1, DKK (Dickkopf) or SFRP (Secreted frizzled-related protein such as SFRP4). Thus, in an embodiment the subject has cells, for example cancer cells, which are heterozygous, more preferably homozygous, for a mutation or epigenetic modification which reduces the activity or levels of a protein involved in Wnt signalling such as, but not necessarily limited to, one or more of APC, TCF7L2 (=TCF4), CTNNB1, WTX, AXIN1, DKK or a SFRP. Examples of disease characterized by excessive Wnt signalling, including high frequency Wnt pathway mutations in colorectal cancer, is provided in Table 1. In another preferred embodiment, the disease is further characterized by normal (non-mutated) gp130/Jak2/stat3 signalling. For instance, this embodiment excludes diseases such as colitis, which are associated with excessive gp130/Jak2/stat3 signalling.

“JAK” as used herein refers to a polypeptide belonging to the Janus Kinase family of tyrosine kinases. Members of the Janus Kinase family relevant to the invention are JAK2, JAK1 and TYK2.

JAK “activity” refers to the phosphorylation of a substrate by a JAK.

By “inhibits” or “inhibiting” the activity of one or more of JAK2, JAK1 and TYK2 is meant a decrease in kinase activity of the enzyme(s) in a cell. The degree of decrease in activity will vary with the nature and quantity of the compound present, but will be evident as, for example, a detectable decrease in the phosphorylation of a substrate by the enzyme; desirably a degree of decrease greater than 10%, 33%, 50%, 75%, 90%, 95% or 99% as compared to the activity in the absence of the compound.

TABLE 1
Cancer types with high frequency of WNT pathway mutations.

| | Mutated WNT | |
|---|---|---|
| Cancer | pathway component | Other evidence |
| Colorectal cancer | APC, TCF7L2 | |
| | (=TCF4), CTNNB1, | |
| | WTX, AXIN1 | |
| Hepatocellular | CTNNB1, AXIN1 | |
| carcinoma | | |
| Medullablastoma | CTNNB1 | |
| Ovarian cancer | CTNNB1 | oversupply of WNT5a, |
| | | WNT7 |
| Pancreatic cancer | APC | |
| Gastric cancer | APC | oversupply of WNT5a |
| Endometrial cancer | CTNNB1 | |
| Adrenocortical cancer | CTNNB1 | |
| Pituitary gland | CTNNB1 | |
| cancer | | |
| Biliary tract cancer | CTNNB1, AXIN1 | |
| Kidney cancer | WTX | |
| Soft tissue cancer | APC, CTNNB1 | |
| Breast cancer | | oversupply of WNT1, |
| | | WNT3A, WNT7A |
| Oesophageal cancer | APC, CTNNB1 | oversupply of WNT2 |
| Gliobalstoma | | high nuclear β-Catenin, |
| | | oversupply of WNT ligand |
| Lung cancer | high nuclear/ | |
| | cytoplasmic | |
| | β-Catenin | |
| Prostate cancer | reduced SFRP4 | high nuclear β-Catenin, |
| | | oversupply of WNT3A, |
| | | WNT5a |
| Leukemia | | nuclear β-Catenin, high |
| (AML, ALL) | | LEF1, Fzd6 (mice) |
| Thyroid cancer | CTNNB1, APC, | |
| | AXIN1 | |

In a preferred embodiment, the compound selectively inhibits one or more of JAK2, JAK1 and TYK2. By “selective” inhibitor is meant a compound that inhibits one or more of JAK2, JAK1 and TYK2 activity to a greater extent when compared to other kinases such as other JAK kinases (for instance JAK3). In one embodiment, the compound may be a selective inhibitor of JAK2 (for example, over JAK1, JAK3 and TYK2). Selectivity can be at least about 5-fold, at least about 10-fold, at least about 20-fold, at least about 50-fold, at least about 100-fold, at least about 200-fold, at least about 500-fold or at least about 1000-fold. Selectivity can be measured by methods routine in the art. In some embodiments, the selectivity of a compound can be determined by cellular assays associated with particular JAK kinase activity.

As used herein, the term “subject” relates to an animal. More preferably, the subject is a mammal such as a human, dog, cat, horse, cow, or sheep. Alternatively, the subject may be avian, for example, poultry such as a chicken, turkey or duck. Most preferably, the subject is a human.

As used herein, the terms “treating”, “treat” or “treatment” include administering a therapeutically effective amount of a compound(s) described herein sufficient to reduce or eliminate at least one symptom of a disease, and/or sufficient to reduce or arrest cancer cell proliferation.

As used herein, the terms “preventing”, “prevent” or “prevention” include administering a therapeutically effective amount of a compound(s) described herein sufficient to stop or hinder the development of at least one symptom of a disease.

### JAK1, JAK2 and TYK2 Inhibitors

As the skilled person would appreciate, a wide range of different compounds can be used for the invention including small chemicals (molecules), proteins which bind and inhibit kinase activity (such as antibodies, peptides or mimetics thereof), and nucleic acid based therapies such as the delivery of double stranded RNA (dsRNA) for gene silencing.

### Small Molecules

WO2009114514 to Incyte discloses substituted pyrrolopyridine and pyrrolopyrimidines of the general structure shown below as JAK inhibitors. The disclosures of WO2009114514 indicate that a preferred 5-membered ring formed by A¹, A², U, T and V is pyrazole.

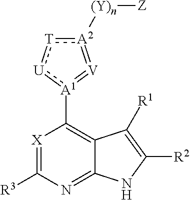

The first JAK inhibitor approved for clinical use was Incyte's Ruxolitinib (Jakafi). Ruxolitinib is shown below.

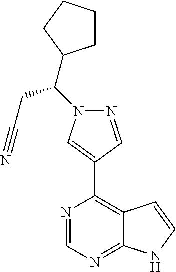 [SMILES: N#CC[C@H](C1CCCC1)n1cc(-c2ncnc3[nH]ccc23)cn1]

WO2009114512, also to Incyte, discloses azetidine and cyclobutane derivatives of the general structure shown below as JAK inhibitors.

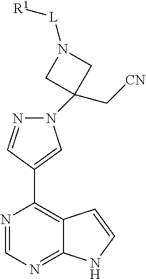

Baricitinib (also known as LY3009104 or INCB28050) is in phase II clinical trials for the treatment of rheumatoid arthritis and diabetic kidney disease. Baricitinib is shown below.

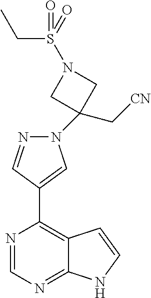 [SMILES: CCS(=O)(=O)N1CC(CC#N)(n2cc(-c3ncnc4[nH]ccc34)cn2)C1]

Further disclosures of inhibitors useful for the invention from Incyte include WO2010135650 and WO2011112662 which describe analogues of Baricitinib with many of the compounds exemplified displaying good JAK1 selectivity and potency. Examples of potent and selective JAK1 compounds as exemplified are shown below (WO2011112662).

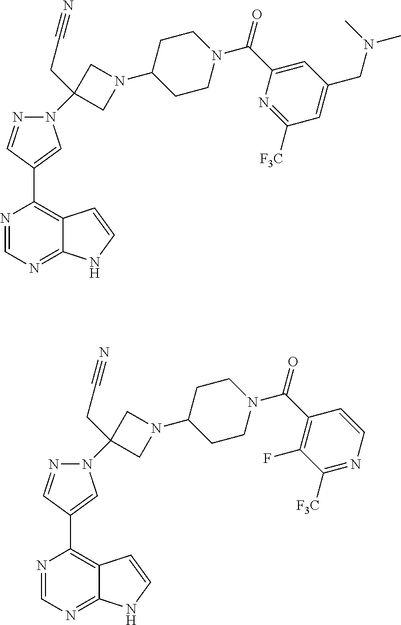 [SMILES: CN(C)Cc1cc(C(=O)N2CCC(N3CC(CC#N)(n4cc(-c5ncnc6[nH]ccc56)cn4)C3)CC2)nc(C(F)(F)F)c1 ; N#CCC1(n2cc(-c3ncnc4[nH]ccc34)cn2)CN(C2CCN(C(=O)c3ccnc(C(F)(F)F)c3F)CC2)C1]

WO2009132202, also to Incyte, discloses macrocyclic compounds of the general formula below for the treatment of various diseases.

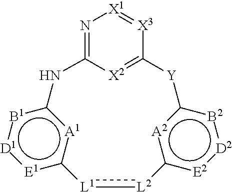

WO2008011174 to Cephalon discloses JAK inhibitors comprising the indolocarbazole general structure as shown below.

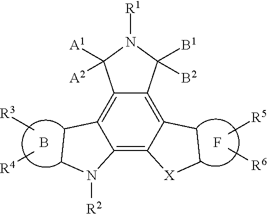

Cephalon's compound Lestaurtinib (also known as CEP701 and first disclosed in WO1998007045) is in ongoing Phase II studies. Lestaurtinib is shown below.

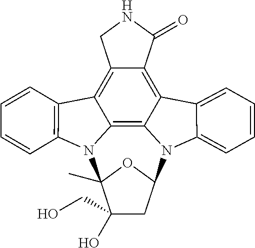 [SMILES: C[C@]12O[C@H](C[C@]1(O)CO)n1c3ccccc3c3c4c(c5c6ccccc6n2c5c31)CNC4=O]

WO2010141796, also to Cephalon, discloses a series of structurally distinct [1,2,4]-triazolo[1,5-a]pyridine derivatives of the general structure shown below.

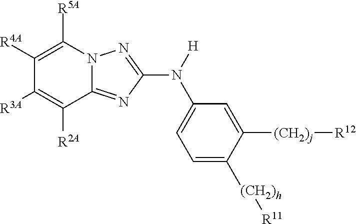

The selective JAK2 inhibitor CEP-33779, shown below, is disclosed in WO2010141796.

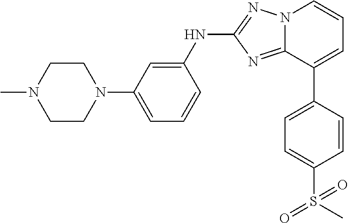 [SMILES: CN1CCN(c2cccc(Nc3nc4c(-c5ccc(S(C)(=O)=O)cc5)cccn4n3)c2)CC1]

WO2007058627 to S*Bio (Cell Therapeutics, Inc.) discloses oxygen linked pyrimidine derivatives of the general structure shown below.

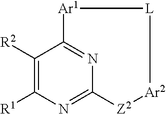

Pacritinib, also known as SB1518, (disclosed in WO2007058627 and in WO2010068181 and WO2010068182 as the citrate and maleate salts respectively) is in phase II clinical trials for the treatment of myelofibrosis. Pacritinib is shown below.

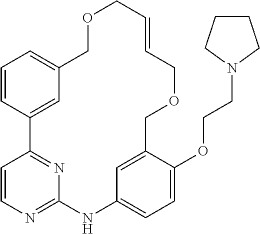 [SMILES: C1=C/COCc2cc(ccc2OCCN2CCCC2)Nc2nccc(n2)-c2cccc(c2)COC/1]

SB1578 (disclosed in WO2007058627 and in WO2011008172 as the citrate salt) is in ongoing phase I studies for the treatment of rheumatoid arthritis. SB1578 is shown below.

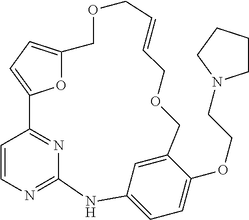 [SMILES: C1=C/COCc2cc(ccc2OCCN2CCCC2)Nc2nccc(n2)-c2ccc(o2)COC/1]

Other macrocyclic inhibitors have been disclosed in WO2011097525. WO2011097525 exemplifies the nitrogen-linked pyrimidine SB1317 shown below.

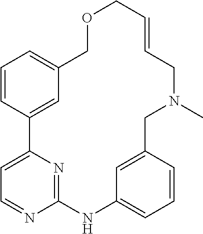 [SMILES: CN1C/C=C/COCc2cccc(c2)-c2ccnc(n2)Nc2cccc(c2)C1]

WO2007053452 to TargeGen, Inc. discloses bi-aryl meta-pyrimidine inhibitors of the general structure shown below.

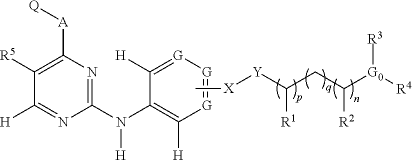

TG101348 (now Sanofi Aventis SAR302503) is selective for JAK2 and is in phase III clinical trials for the treatment of myelofibrosis. TG101348 is shown below.

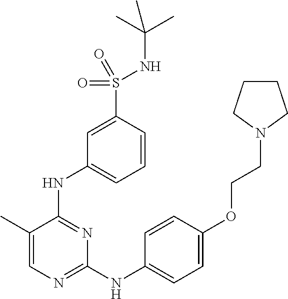 [SMILES: Cc1cnc(Nc2ccc(OCCN3CCCC3)cc2)nc1Nc1cccc(S(=O)(=O)NC(C)(C)C)c1]

TG101209, shown below, is also a potent JAK2 inhibitor.

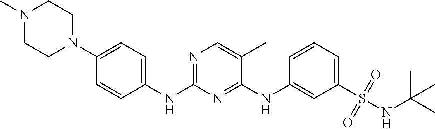 [SMILES: Cc1cnc(Nc2ccc(N3CCN(C)CC3)cc2)nc1Nc1cccc(S(=O)(=O)NC(C)(C)C)c1]

WO2003099811, WO2008109943 and WO2009029998 to Cytopia disclose pyrazine and pyrimidine based compounds. The JAK1/JAK2 selective compound CYT387 (WO2008109943) is in phase I clinical trials for the treatment of myelofibrosis. CYT387 is shown below.

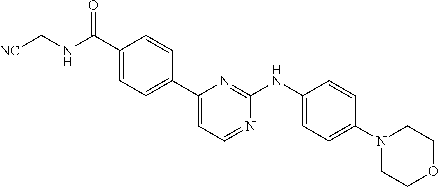 [SMILES: N#CCNC(=O)c1ccc(-c2ccnc(Nc3ccc(N4CCOCC4)cc3)n2)cc1]

WO2007049041 to AstraZeneca discloses 4-(3-aminopyrazole) pyrimidine derivatives of the general structure shown below. AstraZeneca also discloses further Heterocyclic compounds as JAK inhibitors in WO2010038060 and WO2010020810.

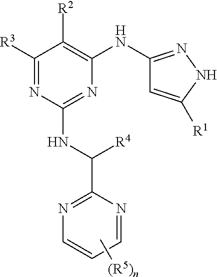

The JAK2 selective compound AZD1480 (WO2007049041) is in phase II clinical trials for the treatment of myelofibrosis. AZD1480 is shown below.

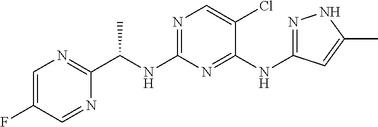 [SMILES: Cc1cc(Nc2nc(N[C@@H](C)c3ncc(F)cn3)ncc2Cl)n[nH]1]

WO2006082392 to AstraZeneca discloses pyrazolylaminopyridine derivatives as kinase inhibitors. WO2006082392 discloses the potent inhibitor of JAK2, AZ960, shown below. Further pyrazolylaminopyridine derivatives with selectivity for JAK2 and TRKs are disclosed in WO2008117050.

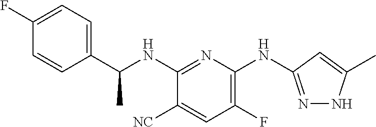 [SMILES: Cc1cc(Nc2nc(N[C@@H](C)c3ccc(F)cc3)c(C#N)cc2F)n[nH]1]

Further pyrazolylaminopyridine derivatives with selectivity for JAK2 and TRKs are disclosed in WO2008117050. The general structure of the compounds claimed in WO2008117050 is shown below. In this general structure, ring A is a heterocyclic ring and ring B is a carbocyclic or heterocyclic ring.

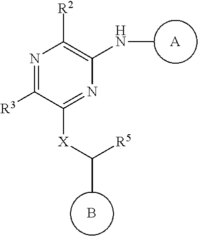

AstraZeneca discloses 4-(3-aminopyrazolyl)-pyrimidines as TRK and JAK2 inhibitors in WO2007049041 and WO2009095712 and further analogues in WO2008135786, WO2008135202, WO2009027736 and WO2009007753. Aminopyrazol-imidazo-pyridine derivatives possessing significant JAK2 and TRK inhibiting potency are disclosed in two further patent applications WO2008129255 and WO2008135785.

WO2010074947 to Eli Lily discloses the selective JAK2 inhibitor LY2784544 as a single compound with no additional exemplification. LY2784544 is in phase II clinical trials for the treatment of myelofibrosis. LY2784544 is shown below.

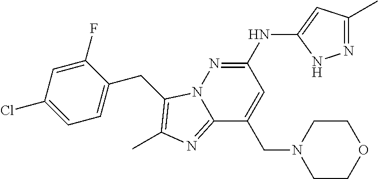 [SMILES: Cc1cc(Nc2cc(CN3CCOCC3)c3nc(C)c(Cc4ccc(Cl)cc4F)n3n2)[nH]n1]

WO2011028864 to Bristol-Myers Squibb discloses imidazo[4,5-c]pyrrolo[2,3-b]pyridines of the general structure shown below as JAK2 inhibitors.

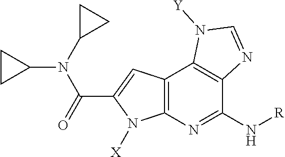

The JAK2 selective compound BMS911543 (WO2011028864) is in phase II clinical trials for the treatment of myelofibrosis. BMS911543 is shown below.

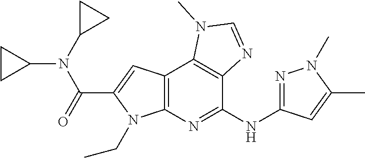 [SMILES: CCn1c(C(=O)N(C2CC2)C2CC2)cc2c3c(ncn3C)c(Nc3cc(C)n(C)n3)nc21]

WO2008106635 to SuperGen, Inc. discloses 2,4-diamino-pyrimidine derivatives as JAK2 inhibitors.

WO2007087246 to Merck discloses MK0457 (also known as the Vertex compound VX680) as a potent JAK2 inhibitor for treating myeloproliferative disorders. MK0457 was initially disclosed by Vertex as an aurora kinase inhibitor (WO2004000833). MK0457 is shown below.

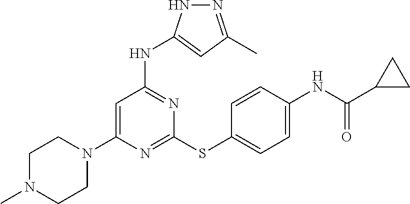 [SMILES: Cc1cc(Nc2cc(N3CCN(C)CC3)nc(Sc3ccc(NC(=O)C4CC4)cc3)n2)[nH]n1]

WO2007056163 and WO2007056164 to Vertex disclose MK0457 analogues.

WO2004000833, WO2007089768 and WO2008042282 to Exelixis Inc. disclose 4-aryl-2-amino-pyrimidines and imidazole-4,5-dicarboxamide derivatives of the general structures shown below as JAK2 modulators.

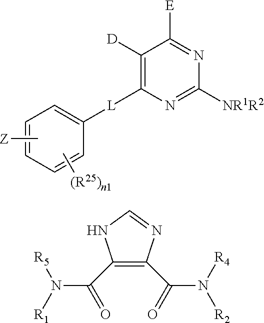

The Exelis compound XL019 (structure undisclosed), with reasonable selectivity for JAK2, was recently reported to successfully complete phase I clinical trials in patients with primary myelofibrosis.

WO2012037132, also to Exelis, discloses 1-anilino-4-phenylphthalazine compounds of the general structure shown below as selective JAK1 inhibitors.

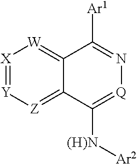

AG490 (shown below), although not specific for JAK2, was the first reported JAK2 inhibitor identified by a high throughput screen (Meydan et al., 1996).

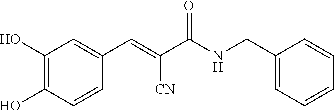 [SMILES: N#C/C(=C\c1ccc(O)c(O)c1)C(=O)NCc1ccccc1]

Several AG490 analogous compounds have been developed with higher affinity and specificity. WO2001079158 discloses LS104 shown below.

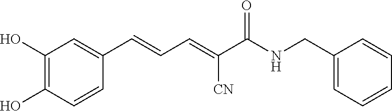 [SMILES: N#C/C(=C\C=C\c1ccc(O)c(O)c1)C(=O)NCc1ccccc1]

WO2006070195 to Astex Therapeuitcs discloses pyrazole compounds of the general structure shown below as kinase inhibitors.

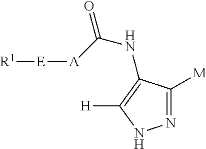

The compound AT9283 is in phase II clinical trials for treating advanced or metastatic solid tumors or Non-Hodskin's Lymphoma. AT9283 is shown below.

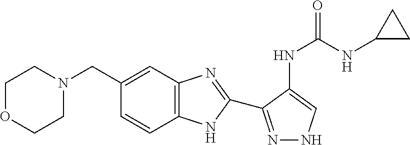 [SMILES: O=C(Nc1c[nH]nc1-c1nc2cc(CN3CCOCC3)ccc2[nH]1)NC1CC1]

Novartis AG has disclosed sulfonamidoanilines (WO2007071393), 2,4-di(arylamino)-pyrimidine-5-carboxamides (WO2008009458), anellated nitrogen heterocycles (WO2008052734), quinoxaline derivatives (WO2008148867) and pyrrolo[2,3-d]pyridines (WO2009098236) as JAK2 inhibitors. NVP-BSK805 (WO2008148867) shown below has been disclosed as a potent JAK2 inhibitor.

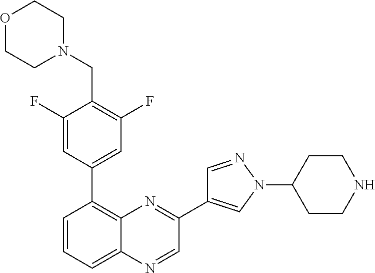 [SMILES: Fc1cc(-c2cccc3ncc(-c4cnn(C5CCNCC5)c4)nc23)cc(F)c1CN1CCOCC1]

WO2008118822 and WO2008118823 to Rigel disclose 2,4-diamino-pyrimidine compounds as JAK2 inhibitors. WO2009103032 discloses analogous 2-amino-pyrimidine compounds as JAK2 inhibitors.

WO2010099379 to Ambit Biosciences discloses quinazoline derivatives of the general structure shown below as JAK2 inhibitors. Further quinazoline analogues are disclosed in WO2012030912, WO2012030914 and WO2012030948.

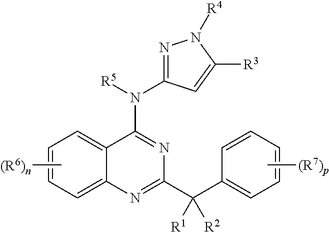

WO2012030885 and WO2012030917 to Ambit Biosciences disclose AC430 for the treatment of JAK-mediated conditions, disorders or diseases. AC430 is shown below.

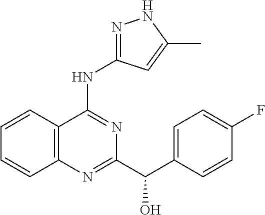 [SMILES: Cc1cc(Nc2nc([C@@H](O)c3ccc(F)cc3)nc3ccccc23)n[nH]1]

WO2010149769 to Galapagos NV discloses [1,2,4]triazolo[1,5-a]pyridine compounds of the general structure shown below as JAK inhibitors. The selective JAK1 inhibitor GLPG0634 (structure undisclosed) is in phase II clinical trials for the treatment of rheumatoid arthritis.

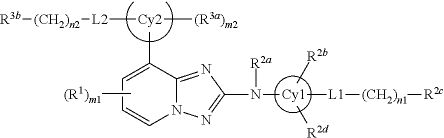

The specific [1,2,4]triazolo[1,5-a]pyridine compound shown below is disclosed in WO2010010184.

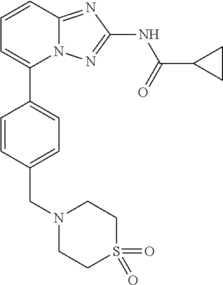 [SMILES: O=C(Nc1nc2cccc(-c3ccc(CN4CCS(=O)(=O)CC4)cc3)n2n1)C1CC1]

WO2009155551 and WO2009155565 to Roche (Genentech) disclose aryl-2-arylamino-[1,2,4]triazolo[1,5-a]pyridines of the general structure below as JAK inhibitors. A subsequent Roche application (WO2010051549) discloses pyrazolo[1,5a]-pyrimidines compounds that are selective for either JAK2 over JAK3 or for JAK3 over JAK2.

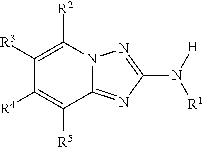

WO2011113802, WO2012035039 and WO2012066061, also to Roche, disclose derivatives of 3H-imidazo[4,5-c]pyridin-4-amines, 7H-purin-6-amines, thiazolo[5,4-c]pyridin-4-amines, thiazolo[4,5-d] pyrimidin-7-amines, 2H-pyrazolo[4,3-c]pyridin-2-amines and 2H-pyrazolo[3,4-d]pyrimidin-4-amines. The general structures which cover the compounds disclosed are shown below. A number of the compounds display high potency and specificity for TYK2.

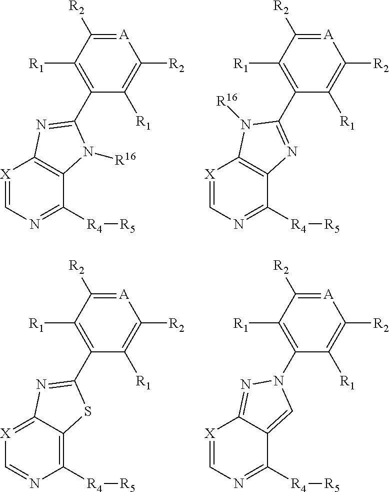

WO2011086053 and WO2012085176 to Roche disclose compounds with tricyclic ring systems (as shown below) as JAK inhibitors with specificity for JAK1.

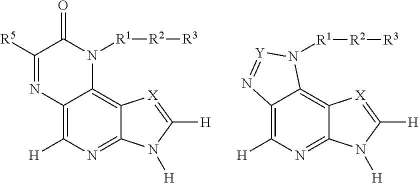

The disclosures of these documents indicate that the scaffold of an imidazopyrrolopyridine ring system, as exemplified in the compound shown below, provides good JAK1 potency and selectivity.

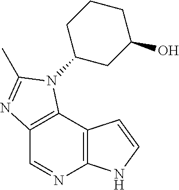 [SMILES: Cc1nc2cnc3[nH]ccc3c2n1[C@@H]1CCC[C@@H](O)C1]

Abbott disclose similar tricyclic compounds as JAK inhibitors in WO2009152133 and WO2011068881.

DE102009015070 to Bayer discloses N-[3-(4-aminopyrimidin-2-yl)aminophenyl] urea derivatives of the general structure shown below as selective inhibitors of TYK2.

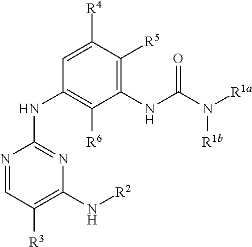

Examples of potent and selective TYK2 compounds disclosed in DE102009015070 are shown below.

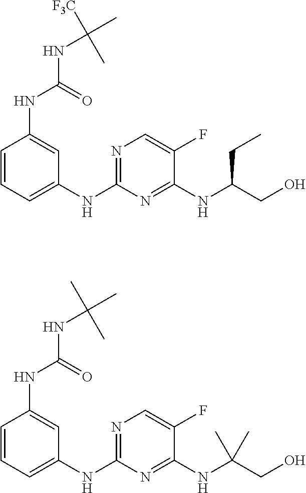 [SMILES: CC[C@@H](CO)Nc1nc(Nc2cccc(NC(=O)NC(C)(C)C(F)(F)F)c2)ncc1F ; CC(C)(C)NC(=O)Nc1cccc(Nc2ncc(F)c(NC(C)(C)CO)n2)c1]

WO2012000970 to Cellzome discloses 5-phenyl-[1,2,4]triazolo[1,5-a]pyridin-2-amine derivatives of the general structure shown below as TYK2 specific inhibitors.

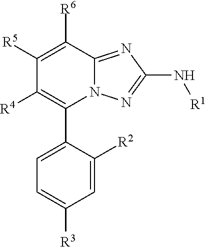

WO2012062704, also to Cellzome, discloses 2-aminopyrimidine, 2-amino-1,3,5-triazine and 2-aminopyridine derivatives of the general structure shown below as TYK2 specific inhibitors. All of the claimed compounds are 4-aryl-2-aminopyrimidine derivatives, with the majority of these being substituted 4-phenyl-2-(pyrazol-4-2-(pyrazol-4-ylamino)pyrimidines which display at least 10-fold selectivity for TYK2over JAK2.

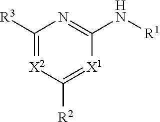

The compound shown below displayed >100-fold selectivity for TYK2.

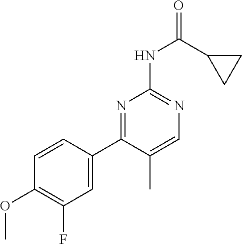 [SMILES: COc1ccc(-c2nc(NC(=O)C3CC3)ncc2C)cc1F]

The following documents also disclose compounds which may be effective generally as JAK inhibitors or as specific inhibitors of one or more forms of JAK: WO2005058829, WO2003030895, WO2004032911, WO2005092904, WO2003068157, WO1998006391, WO2003011285, WO2009035575, WO2007041130, WO2007117494, WO2007084557, WO2008079521, WO2009085913, WO2009046416, WO2007058628, WO2008060248, WO2008140419, WO2008140420, WO2008140421, WO2008054292, WO2007053776, WO2008021369, WO2008047831, WO2008154241, WO2008157207, WO2009017954, WO2009106441, WO2009106443 and WO2002078617.

### Antibodies

In one embodiment, the compound is an antibody.

The term “antibody” as used herein includes polyclonal antibodies, monoclonal antibodies, bispecific antibodies, diabodies, triabodies, heteroconjugate antibodies, chimeric antibodies including intact molecules as well as fragments thereof, and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain.

A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing al chimeric Fv region. The antibody may be of animal (for example mouse, rabbit or rat) or human origin or may be chimeric (Morrison et al., 1984) or humanized (Jones et al., 1986).

As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Such antigen binding agents can be made as described by Harmsen and De Haard (2007), Tibary et al. (2007), and Muyldermans (2001), and references cited therein. Yeast SPLINT antibody libraries are available for testing for intrabodies which are able to disrupt protein-protein interactions (see for example, Visintin et al., 2008a and Visintin et al, 2008b for methods for their production). Such agents may comprise cell-penetrating peptide sequence or nuclear-localizing peptide sequence such as those disclosed in Constantini et al. (2008). Also useful for in vivo delivery are Vectocell or Diato peptide vectors such as those disclosed in De Coupade et al. (2005) and Meyer-Losic et al. (2006).

In addition, the antibodies may be fused to a cell penetrating agent, for example a cell-penetrating peptide. Cell penetrating peptides include Tat peptides, Penetratin, short amphipathic peptides such as those from the Pep-and MPG-families, oligoarginine and oligolysine. In one example, the cell penetrating peptide is also conjugated to a lipid (C6-C18 fatty acid) domain to improve intracellular delivery (Koppelhus et al., 2008). Examples of cell penetrating peptides can be found in Howl et al., (2007) and Deshayes et al. (2008). Thus, the invention also provides the therapeutic use of antibodies fused via a covalent bond (e.g. a peptide bond), at optionally the N-terminus or the C-terminus, to a cell-penetrating peptide sequence.

Antibodies which inhibit one or more of JAK1, JAK2 or TYK2 activity are available from various sources such as Santa Cruz Biotechnology.

### Polynucleotides

In a further embodiment, one or more of JAK1, JAK2 or TYK2 activity in a cell of the subject is inhibited by the delivery of a polynucleotide which results in a reduction in the production levels of one or more of JAK1, JAK2 or TYK2. The polynucleotide may be delivered by any means known in the art such as, but not limited to, administration of the polynucleotide per se, or through the administration of a vector (such as a virus) expressing the polynucleotide. Examples of such polynucleotides include, but are not limited to, antisense polynucleotides, catalytic polynucleotides, microRNAs, and double-stranded RNA molecules such as siRNAs and shRNAs.

### Antisense Polynucleotides

The term “antisense polynucleotide” shall be taken to mean a DNA or RNA, or combination thereof, molecule that is complementary to at least a portion of a specific mRNA molecule encoding a polypeptide and capable of interfering with a post-transcriptional event such as mRNA translation. The use of antisense methods is well known in the art (see for example, G. Hartmann and S. Endres, Manual of Antisense Methodology, Kluwer (1999)).

An antisense polynucleotide useful for the invention will hybridize to a target polynucleotide under physiological conditions. As used herein, the term “an antisense polynucleotide which hybridises under physiological conditions” means that the polynucleotide (which is fully or partially single stranded) is at least capable of forming a double-stranded polynucleotide with mRNA encoding a protein, in a cell.

Antisense molecules may include sequences that correspond to the structural genes or for sequences that effect control over the gene expression or splicing event. For example, the antisense sequence may correspond to the targeted coding region of the target gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Perriman et al., 1992) and the hairpin ribozyme (Shippy et al., 1999).

The ribozymes useful for this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides useful for the invention should also be capable of hybridizing a target nucleic acid molecule under “physiological conditions”, namely those conditions within a cell (especially conditions in an animal cell such as a human cell).

### RNA Interference

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

The methods of the present invention utilise nucleic acid molecules comprising and/or encoding double-stranded regions for RNA interference. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules as described herein can be used to epigenetically silence genes at both the post-transcriptional level or the pre-transcriptional level. In a non-limiting example, epigenetic regulation of gene expression by siRNA molecules as described herein can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl- adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

microRNA

MicroRNA regulation is a specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Screening Methods

As outlined above, in an embodiment the method of the invention further comprises testing the subject to determine whether they have a disease characterized by excessive Wnt signalling before administering the compound. As the skilled person will appreciate, such testing can be conducted in a variety of ways. Typically, the testing will involve obtaining a biological sample (for example, blood, saliva or hair follicles) from the subject comprising nucleic acids, preferably genomic DNA, and analysing the sample for the one or more mutations or epigenetic modifications. In one embodiment, the sample comprises cancerous cells.

Somatic mutations (including small deletions and insertions, missense and non-sense mutation, as well as chromosome breaks and Loss-of heterozygosity) in components of the canonical Wnt signaling pathway associated with human cancers have been compiled in http://cancer.sanger.ac.uk/cancergenome/proiects/cosmic/. Examples of common mutations include, but are not limited to:

### APC

Mutation id: COSM18862

AA Mutation: p.Q1378* (Substitution-Nonsense)

CDS Mutation: c.4132C>T (Substitution)

Mutation id: COSM19020

AA Mutation: p.T1556fs*3 (Insertion-Frameshift)

CDS Mutation: c.4665_{—}4666insA (Insertion)

Mutation id: COSM18561

AA Mutation: p.T1556fs*3 (Insertion-Frameshift)

CDS Mutation: c.4666_{—}4667insA (Insertion)

### CTNNB1

Mutation id: COSM5664

AA Mutation: p.T41A (Substitution-Missense)

CDS Mutation: c.121A>G (Substitution)

Mutation id: COSM5667

AA Mutation: p.S45F (Substitution-Missense )

CDS Mutation: C.1340>T (Substitution)

### AXIN1

Mutation id: COSM143841

AA Mutation: p.W85* (Substitution-Nonsense )

CDS Mutation: c.254G>A (Substitution)

Nucleic acids can be analysed by a variety of procedures, however, typically genetic assays will be performed. Genetic assay methods include the standard techniques of analysis of methylation patterns, restriction fragment length polymorphism assays, sequencing and PCR-based assays (including multiplex F-PCR STR analysis, whole genome amplification. RT-PCR, digital PCR, and microarray analysis), as well as other methods described below.

The genetic assays may involve any suitable method for identifying mutations, polymorphisms or epigenetic modifications, such as: sequencing of the nucleic acids at one or more of the relevant positions; differential hybridisation of an oligonucleotide probe designed to hybridise at the relevant positions of either the wild-type or mutant sequence; denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; selective DNA amplification using oligonucleotides which are matched for the wild-type sequence and unmatched for the mutant sequence or vice versa; or the selective introduction of a restriction site using a PCR (or similar) primer matched for the wild-type or mutant genotype, followed by a restriction digest. The assay may be indirect, ie capable of detecting a mutation at another position or gene which is known to be linked to one or more of the mutant positions. The probes and primers may be fragments of DNA isolated from nature or may be synthetic.

A non-denaturing gel may be used to detect differing lengths of fragments resulting from digestion with an appropriate restriction enzyme. The DNA is usually amplified before digestion, for example using the polymerase chain reaction (PCR) method and modifications thereof.

Amplification of nucleic acids may be achieved by the established PCR methods or by developments thereof or alternatives such as quantitative PCR, quantitative fluorescent PCR (QF-PCR), multiplex ligation dependent probe amplification, digital PCR, RT-PCR, restriction fragment length polymorphism PCR (PCR-RFLP), PCR-RFLP/RT-PCR-RFLP, hot start PCR, nested PCR, in situ polonony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In another method, a pair of PCR primers are used which hybridise to either the wild-type genotype or the mutant genotype but not both. Whether amplified DNA is produced will then indicate the wild-type or mutant genotype (and hence phenotype).

A preferable method employs similar PCR primers but, as well as hybridising to only one of the wild-type or mutant sequences, they introduce a restriction site which is not otherwise there in either the wild-type or mutant sequences.

In order to facilitate subsequent cloning of amplified sequences, primers may have restriction enzyme sites appended to their 5′ ends. Thus, all nucleotides of the primers are derived from the gene sequence of interest or sequences adjacent to that gene except the few nucleotides necessary to form a restriction enzyme site. Such enzymes and sites are well known in the art. The primers themselves can be synthesized using techniques which are well known in the art. Generally, the primers can be made using synthesizing machines which are commercially available.

PCR techniques that utilize fluorescent dyes may also be used to detect genetic defects in nucleic acids. These include, but are not limited to, the following five techniques.

i) Fluorescent dyes can be used to detect specific PCR amplified double stranded DNA product (e.g. ethidium bromide, or SYBR Green I).

ii) The 5′ nuclease (TaqMan) assay can be used which utilizes a specially constructed primer whose fluorescence is quenched until it is released by the nuclease activity of the Taq DNA polymerase during extension of the PCR product.

iii) Assays based on Molecular Beacon technology can be used which rely on a specially constructed oligonucleotide that when self-hybridized quenches fluorescence (fluorescent dye and quencher molecule are adjacent). Upon hybridization to a specific amplified PCR product, fluorescence is increased due to separation of the quencher from the fluorescent molecule.

iv) Assays based on Amplifluor (Intergen) technology can be used which utilize specially prepared primers, where again fluorescence is quenched due to self-hybridization. In this case, fluorescence is released during PCR amplification by extension through the primer sequence, which results in the separation of fluorescent and quencher molecules.

v) Assays that rely on an increase in fluorescence resonance energy transfer can be used which utilize two specially designed adjacent primers, which have different fluorochromes on their ends. When these primers anneal to a specific PCR amplified product, the two fluorochromes are brought together. The excitation of one fluorochrome results in an increase in fluorescence of the other fluorochrome.

Examples of methods which can be used to detect one or more mutations or epigenetic modifications in a gene encoding a protein involved in Wnt signalling include, but are not limited to, those described in;

i) Varesco et al. (1993) and Traverso et al. (2002) which relate to detecting mutations/modifications of the APC gene;

ii) Scheel et al. (2010) which relate to detecting mutations/modifications of the WTX gene;

iii) Hazra et al. (2008) and Slattery et al. (2008) which relate to detecting mutations/modifications of the TCF7L2 gene;

iv) Lovig et al. (2002), Durand et al. (2011) and Lazar et al. (2008) which relate to detecting mutations/modifications of the CTNNB1 gene,

v) Jin et al. (2003) and Pan et al. (2008) which relate to detecting mutations/modifications of the AXTN1 gene; and

vi) Lin et al. (2011) which relate to detecting mutations/modifications of the SFRP4 gene.

### Pharmaceutical Compositions, Dosages, And Routes Of Administration

The compounds used in the methods of the invention are typically incorporated into pharmaceutical compositions before administration to the subject. Such compositions typically include the compound and a pharmaceutically acceptable carrier. As used herein, the term “pharmaceutically acceptable carrier” includes any and all solvents (such as phosphate buffered saline buffers, water, saline) dispersion media, coatings, antibacterial and antifungal agents, isotonic and absorption delaying agents, and the like, compatible with pharmaceutical administration. Supplementary active compounds can also be incorporated into the compositions. The use of such media and agents for pharmaceutically active substances is well known in the art. Formulations (compositions) are described in a number of sources that are well known and readily available to those skilled in the art. For example, Remington's Pharmaceutical Sciences (Martin E. W., Easton Pa., Mack Publishing Company, 19th ed., 1995) describes formulations which can be used in connection with the invention.

A pharmaceutical composition is formulated to be compatible with its intended route of administration, e.g., local or systemic. Examples of routes of administration include parenteral, e.g., intravenous, intradermal, subcutaneous, oral (e.g., inhalation), nasal, topical, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent that delays absorption, such as aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the compound into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Formulations suitable for nasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the compound. For administration by inhalation, the compound(s) can also be delivered in the form of drops or an aerosol spray from a pressured container or dispenser that contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, drops, or suppositories. For transdermal administration, the active compound(s) are formulated into ointments, salves, gels, or creams, as generally known in the art.

The pharmaceutical compositions can also be prepared in the form of suppositories (e.g., with conventional suppository bases such as cocoa butter and other glycerides) or retention enemas for rectal delivery.

In another embodiment, the compound is formulated in liposomes. Such formulations can enhance cellular uptake of the compound. Liposomes containing the compound can be prepared by methods known in the art, such as described in U.S. Pat. No. 4,485,045, U.S. Pat. No. 4,544,545 and U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes can be extruded through filters of defined pore size to yield liposomes with the desired diameter.

In accordance with the invention, treatment of a subject with a therapeutically effective amount of the compound can include a single treatment or can include a series of treatments. The compounds can be administered on any appropriate schedule, e.g., from one or more times per day to one or more times per week; including once every other day, for any number of days or weeks, e.g., 1 day, 2 days, 3 days, 4 days, 5 days, 6 days, 1 week, 10 days, 2 weeks, 3 weeks, 4 weeks, 5 weeks, 6 weeks, 7 weeks, 8weeks, 2 months, 3 months, 6 months, or more, or any variation thereon. The skilled artisan will appreciate that certain factors may influence the dosage and timing required to effectively treat a subject, including but not limited to the severity of the disease or disorder, previous treatments, the general health and/or age of the subject, and other diseases present.

The compound(s) used in the compositions and methods of the invention can be used in the form of salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Salts may also be formed with bases. Such salts include salts derived from inorganic or organic bases, for example alkali metal salts such as magnesium or calcium salts, and organic amine salts such as morpholine, piperidine, dimethylamine or diethylamine salts.

In an embodiment, the method of the invention is combined with the use of other methods of treating cancer such as, but not limited to, radiation therapy or chemotherapy regimen. In this instance, without wishing to be limited by theory, the method of the invention may restrict the effect of oncogenic WNT signaling arresting cell division and standard of care chemotherapy used to eliminate the remaining presumptive tumour re-initiating cells. Examples of the other types of chemotherapy compounds which may be used include, but are not limited to, cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), matrixmetalloprotease inhibitors, telomerase inhibitors, tyrosine kinase inhibitors, anti-growth factor receptor agents, anti-HER agents, anti-EGFR agents, anti-angiogenesis agents (e.g. angiogenesis inhibitors), farnesyl transferase inhibitors, ras-raf signal transduction pathway inhibitors, cell cycle inhibitors, cdks inhibitors, tubulin binding agents, topoisomerase I inhibitors, topoisomerase II inhibitors, and the like.

### EXAMPLES

### Example 1

### Inhibiting gp130/stat3 pathway through the activity of one or more gp130-associated jak tyrosine kinases

### Materials and Methods

### Mice

All procedures involving animals were approved by the Ludwig Institute for Cancer Research/Department of Surgery Ethics Committee. Unless indicated, all mice were on an inbred C57/B6 genetic background using appropriate littermates as controls. Post mortem tissue collection and processing was carried out as previously detailed (Bollrath et al., 2009).

### Irradiation Induced Regeneration

Mice were irradiated with a single dose of 14 Gy of γ-irradiation (0.414 Gy/min) to determine regenerative potential of the small intestine as described (Ashton et al., 2010).

### Tumor Xenografts

One flank of female BALB/c nude mice was injected subcutaneously with 2×10⁶ SW480 cells resuspended in Matrigel (BD Biosciences)/PBS (1:1) at a final volume of 200 μl. Once tumor became palpable (>100 mm³) approximately 5 days later, mice received continuous daily gavage of the Jak2 inhibitor AZD1480 (Hedvat et al., 2009) (30 mg/kg, a gift from Astra-Zeneca).

### Organoid Culture

Crypts from small intestines were harvested, washed and were resuspended at 2000 crypts/ml of matrigel (BD Biosciences) and 50 μL was dispensed in each well of a 24-well plate. Once the matrigel had set, medium was added and the organoids cultured for seven days as described (Sato et al., 2009). Experiments were performed in triplicates and repeated.

Organoid cultures were imaged on a Nikon Ti-E microscope using DIC contrast with a 10x PlanApo NA0.3 objective. A focal stack of images was collected (10 μm apart) and processed through the Best Focus function of MetaMorph v7.7.7 (Molecular Devices, USA) to generate the final image of individual Organoids.

### Tissue Culture and Soft Agar Colony Growth Assay

SW480 and SW480^{APC} cells (Faux et al., 2004) were seeded in 2 ml of RPMI media supplemented with 10% FBS and 1% penicillin/streptomycin in 35 mm culture dishes at a density of 2×105 cells per dish. Three days later cultures were exposed to AZD1480 (1 μM) for 15 min. Co-immunofluorescence was then performed with antibodies to rabbit phospho-Stat3 (Tyr705, Santa Cruz #9134s, 1:200 dilution) and mouse β-catenin (Transduction Laboratories, 1:400). Colony formation assays of SW480 cells in soft agar were performed as described (Faux et al., 2004) in the presence of either AZD1480, (1 μM), the Stat3 inhibitor S3I-201 (Biovision, 50 μM) or recombinant human IL11 (Genetics Institute, 10 ng/ml). All experiments were performed in triplicates and repeated at least once. Vehicle was methylcellulose.

### Luciferase Reporter Assay

A 550 bp region around the predicted Stat3 binding site in the first intron of the murine Bmi1 gene (Vallania et al., 2009) was amplified from genomic DNA using the primers (F) 5′aagctcgagagggtttaagcaccttg3′ (SEQ ID NO: 1) and (R) 5′aagagatctcccaaacctgcagcaactat3′ (SEQ ID NO: 2) and subcloned into pGL4_{—}23[luc2/minP] (Promega). For luciferase assays, 293T cells were seeded in 96 well plates at 1×10⁴ cells/well the day before transfection in DMEM supplemented with 10% FBS. The pBmi1:luc2 and pCMV-renilla plasmids were co-transfected at a ratio of 40:1 using FuGENE 6 transfection reagent (Roche). The next day, cells were stimulated with the indicated concentrations of Hyper IL-6 (Ernst et al., 2008), for 4 hr before cell cultures were processed using the Dual-Luciferase Reporter Assay (Promega) and luminescence was measured using a Lumistar Galaxy luminometer (Dynatech Laboratories). Experiments were performed in triplicates.

### Expression Analysis

Gene expression analysis by RT-qPCR was performed using the SybrGreen method (ABI) as described previously (Phesse et al., 2008). Fold change was calculated using the 2^{-ΔΔCT} method (Livak et al., 2001). Protein expression was analysed by Western blot by transferring SDS-PAGE separated protein lysates to nitrocellulose membrane and blocked either in 5% non-fat milk powder and probed with antibodies detecting p16 (Santa Cruz; #sc-467), Bmi1 (Millipore #??), Actin (Sigma; #A2066) or Gapdh (Santa Cruz; #sc-69778). All antibodies were diluted 1:1000 in BSA or non-fat milk powder and incubations were performed overnight.

### Immunohistochemistry

Tissues were fixed and immunohistochemistry performed as described previously (Sansom et al., 2007). Primary antibodies used were rabbit phospho-Stat3 to Tyr705 (Santa Cruz #9134s, 1:150), mouse β-catenin (Transduction Laboratories, 1:300), rabbit PCNA (Santa Cruz #7907, 1:100), rabbit β-Myc (Santa Cruz N-262. Lot #C1309, 1:200), and goat p21 (Santa Cruz #Sc-397-G; 1:100).

### Histology

Hematoxilin and Eosin-stained sections were scanned using an Aperio ScanScope XT (Aperio, USA) pathology slide scanner with a 20x PlanApo NA0.6 objective and areas of interest were extracted using Aperio ImageScope software v11.1.2.760. The area of individual tumors was outlined using MetaMorph v7.7.7 and standardized against the entire length of the colon section as described (Barker et al., 2009).

To assess the distribution of PCNA and β-catenin the inventors co-stained sections of mouse intestine using primary antibodies detailed above and fluorescent secondaries (anti-rabbit Alexa-488 for PCNA and anti-mouse Alexa-546 for β-catenin). Slides were then tile scanned on a Nikon Ti-E microscope using a 10x PlanApo NA0.3 objective, and the area of individual tumours was outlined manually to extract the nuclei for the generation of binary masks using the count nuclei app module in MetaMorph v7.7.7. Staining of PCNA and β-catenin was determined from these binary masks by measuring area, average intensity, shape factor and intensity standard deviation. Resulting values were assembled in Excel files and analysed with FloJo to determine the number of cells with nuclei staining positive for either of the two molecules.

### Statistics

Unless indicated otherwise, values were statistically assessed using the Mann-Whitney U test for non-parametric analysis of independent observations, and values are depicted as the Mean±SEM.

### Results and Discussion

The present inventors have surprisingly found that intestinal regeneration was completely blocked in gp130^{ΔStat/ΔStat} mice where gp130 cytokines are unable to activate Stat3 (FIG. 1a and b). Likewise, gp130^{ΔStat/+} and Stat3^{+/-} mice showed reduced intestinal regeneration compared to wild-type mice (FIG. 1a and b). Conversely, excessive gp130/Stat3 signalling enhanced intestinal regeneration of gp130^{F/F} mice through a Jak2- and Stat3-dependent mechanism, since regeneration was impaired in gp130^{F/F} ;Stat3^{+/-} mice or in gp130^{F/F} mice treated with the Jak2-specific inhibitor AZD1480 (Hedvat et al, 2009) (FIG. 1a and b).

The proliferative potential of the crypt stem cell compartment underpins the intestines ability to rapidly regenerate and is manifested by the capacity of cultured intestinal organoids to form crypt-like outgrowth (Sato et al., 2011). The present inventors observed that crypt formation was greater in organoids derived from gp130^{F/F} than from wild-type mice (FIG. 1c and d). Conversely, crypt formation was reduced in gp130^{F/F} organoids grown in the presence of the Stat3 antagonist S3I-201 (Siddiquee et al., 2007 (FIG. 1c and d). These results suggest that gp130/Stat3 signalling is likely to stimulate crypt regeneration in vivo also through IEC-autonomous mechanisms, independent of the DNA damage induced by gamma irradiation, and suggest that intestinal regeneration may require functional co-operation between Wnt and gp130/Stat3 signalling.

The transcription factor c-myc is a transcriptional target of the Wnt/β-catenin pathway and is required for intestinal regeneration (Ashton et al., 2010). The inventors monitored for nuclear accumulation of c-myc and β-catenin as indicators of active Wnt signalling, and observed extensive staining for both markers throughout the stem cell compartment of regenerating crypts in wild-type mice (FIG. 1e). Remarkably, the inventors also observed their nuclear accumulation in the stunted and non-regenerating crypts of gp130^{ΔStat/+} and of Stat3^{+/-} mice (FIG. 1e and data not shown). Likewise, the bona fide β-catenin target genes Lgr5, Fzd7, Axin2 and CD44 (Phesse et al., 2008) remained similarly elevated in epithelium of irradiated gp130^{ΔStat/+} and wild-type mice (FIG. 1f). By contrast, expression of the cell cycle regulator cycd1, a shared target gene for β-catenin and Stat3, was reduced in gp130^{ΔStat/+} mice (FIG. 1f). These data suggests that Wnt/β-catenin signalling is incapable of promoting regeneration in the absence of sufficient gp130/Jak2/Stat3 signalling.

Aberrant activation of the Wnt/β-catenin pathway is the initiating event in the majority of sporadic colorectal cancer (CRC) and remains essential for sustained tumour promotion and metastatic spread thereafter (Sansom et al., 2004; Barker et al., 2009; Fodde and Brabletz, 2007; Klaus and Birchmeier, 2007). To determine whether gp130/Stat3 signalling also impacts on Wnt/β-catenin dependent tumour formation, the inventors investigated the Apc^{Min/+} model of familial adenomatous polyposis, where tumours arise upon loss of the remaining Apc wild-type allele. Methylene blue stains revealed that 3 month old Apc^{Min/+};gp130^{F/F} mice carried more and larger tumours than their Apc^{Min/+} littermates (FIG. 3a-c). Conversely, genetic Stat3 inhibition in Apc^{Min/+};Stat3^{+/-} mice, or gp130-dependent Stat3 activation in Apc^{Min/+};gp130^{ΔStat/+} mice, reduced tumour number and size (FIG. 2a-c and FIG. 3a,b). However, unlike camplete IEC-specific Stat3 ablation which promoted progression to invasive adenocarcinomas in Apc^{Min/+};Stat3^{ΔIEC/ΔIEC} mice (Musteanu et al., 2010), tumours in Apc^{Min/+};gp130^{ΔStat/+} mice remained as tubular adenomas (FIG. 4c).

To eliminate the possibility that Stat3 modified the kinetics of loss-of-heterozygosity in Apc^{Min/+} mice, the inventors simultaneously inactivated both copies of Apc in adult Lgr5^{CreERT2};Apc^{fl/fl} mice in response to tamoxifen-dependent Cre activation in intestinal stem cells. As reported, tamoxifen administration induced copious tumour formation in the small and large intestine (Barker et al., 2009), but tumour development was almost completely prevented by genetic suppression of gp130-dependent Stat3 activation in Lgr5^{CreERT2};Apc^{fl/fl};gp130^{ΔStat/+} and Lrg5^{CreERT2}; Apc^{fl/fl}; Stat3^{flox/+} mice (FIG. 2d and e). Importantly, prophylactic administration of the Jak2 inhibitor AZD1480 also profoundly reduced growth and de novo tumour formation in tamoxifen-treated Lgr5^{CreERT2};Apc^{fl/fl} mice (FIG. 3d and e). To explore the potential therapeutical benefit from gp130/Jak2/Stat3 pathway inhibition, the inventors commenced treatment of 6 week old Apc^{Min} mice with established intestinal tumours (FIG. 2f and g). While overall tumour burden increased over the next 6 weeks in the vehicle-control cohort, tumour number and total tumour area remained remarkably constant in the AZD1480-cohort (FIG. 2f and g). These data therefore demonstrate that interference with gp130/Jak2/Stat3 signalling restrains Wnt-dependent tumour growth as well as de novo tumour formation.

To show formally that deregulated Wnt signalling was insufficient to promote proliferation of tumours cells with reduced gp130/Jak2/Stat3 signalling, the inventors performed co-immunofluorescence for β-catenin and the proliferation marker PCNA. It was observed that the majority of the abundant β-catenin positive cells in the large Lgr5^{CreERT2};Apc^{fl/fl} tumours also stained for PCNA (FIG. 4a, b and c). In contrast, the proportion of double positive β-catenin/PCNA cells in the few emerging neoplastic foci of Lgr5^{CreERT2};Apc^{fl/fl};gp130^{ΔStat/+} mice was significantly lower even though Apc deletion was induced in both strains at the same time (FIG. 4a, b and c).

The inventors next performed colony assays with SW480 cells to determine whether gp130/Stat3 signalling also impacts on human CRC cells with a mutant WNT/β-CATENIN pathway. The lack of functional APC in SW480 cells results in nuclear β-CATENIN accumulation, colony formation in soft agar and tumour xenograft growth in mice, whereas isogenic SW480^{APC} cells, engineered to express wild-type APC, lack these properties (Faux et al., 2004). Exposure of either cell line to AZD1480 reduced accumulation of activated (phosphorylated) p-STAT3 and reduced SOCS3 expression, but did not diminish accumulation of nuclear β-CATENIN in APC-deficient SW480 cells (FIG. 4b). Despite excessive WNT signalling in these cells, their capacity to form colonies was strongly impaired in the presence of AZD1480 or the STAT3 inhibitor S3I-201 (FIG. 4e and f). Strikingly, these inhibitors did not affect colony formation of APC proficient SW480^{APC} cells, suggesting that GP130/JAK2/STAT3 signalling becomes rate limiting only in situations of excessive WNT signalling (FIG. 4e and f). By contrast, activation of gp130/Stat3 signalling in response to the gp130 cytokine IL-11 increased SOCS3 expression and enhanced colony formation of SW480 and SW480^{APC} cells (FIG. 4e and f). RT-qPCR was performed for the Stat3 target gene SOCS3 on the colonies to demonstrate that both cell lines responded to the treatments (FIG. 4g). The inventors extended these observations to xenografts experiments, and found that systemic AZD1480 administration reduced the growth of established SW480 tumours (FIG. 5a and b). Since the inventors used immuno-compromised hosts, the therapeutic effect of Jak2 inhibition is not due to restoring the host's anti-tumour immune response that is suppressed by excessive Stat3 activity (Yu et al., 2009).

A second cell line which exhibits high Wnt signaling, DLD1 CRC cells, was used in xenograft experiments. Systemic AZD1480 administration was also sufficient to reduce xenograft growth in these cells, supporting the observations using SW480 cells in xenografts (FIG. 5c). RKO colorectal cancer cells were also used in xenograft experiments. RKO cells do not contain activating mutations to the Wnt pathway and consequently have comparatively low Wnt signaling (Ou et al., 2011). In contrast to SW480 cells and DLD1 cells (which have high Wnt signaling), treatment of RKO xenografts did not result in reduced tumor growth (FIG. 5d). Collectively, these data demonstrate that cell intrinsic gp130/Jak2/Stat3 signalling becomes rate-limiting for the proliferation of normal and mutant IECs in situations where excessive Wnt pathway activation promotes tumour growth and regeneration.

The inventors also observed elevated expression of senescence markers p16 and p21 in tumours of Apc^{Min/+};gp130^{ΔStat/+} and of Lgr5^{CreERT2};Apc^{fl/fl};gp130^{ΔStat/+} mice (FIG. 6a, b and c). Compared to SW480 xenografts from vehicle-treated animals, p21 expression was also increased in tumours recovered from AZD1480-treated mice (FIG. 6d). RT-qPCR on RNA extracted from SW480 xenografts confirmed that p21 expression was increased in AZD1480 treated mice, and associated with a decrease in expression of the Stat3 target gene SOCS3 and the cell cycle regulator CYCLIND1 (FIG. 6e). Since the polycomb family protein Bmi1 transcriptionally represses these cell cycle inhibitors (Bracken et al., 2007), the inventors investigated whether gp130/Jak2/Stat3 signalling regulates Bmi1 expression. The inventors observed that, similar to Socs3, Bmi1 was rapidly induced in IECs from mice challenged with recombinant IL-11 (FIG. 7a). Furthermore, Bmi1 protein was more abundant in regenerating crypts of γ-irradiated gp130^{F/F} mice than of wild-type mice (FIG. 7b). These observations are consistent with two evolutionarily conserved Stat3 consensus binding sites in intron 1 of the Bmi1 gene that the inventors identified by computational analysis (Vallania et al., 2009) and which conferred gp130 ligand-dependent transcriptional activity of the corresponding p(Bmi1:luc) reporter construct (FIG. 7c). Importantly, the inventors functionally corroborated the requirement for Bmi1 during tumour growth by establishing that Apc^{Min/+}; Bmi1^{+/-}mice developed fewer large tumours (FIG. 7c) and that these tumours had elevated p16 and p21 expression relative to tumours from Bmi1-proficient Apc^{Min/+} mice (FIG. 6a and c).

Linking the regenerative capacity of intestinal crypts to a “gp130/Stat3 rheostat” appears phylogenetically conserved as it is also required for regeneration of the fly mid-gut (Cordero et al., 2012). It affords the intestine with a mechanism to confine rapid mucosal regeneration to sites of greatest inflammation and, when impaired in gp130^{ΔStat/ΔStat} mice, results in ulcerations at intestinal sphincters exposed to sustained mechanical trauma (Ernst et al., 2001). Here the inventors show that this mechanism, when hijacked by APC-mutant cells, also fuels tumour growth. Importantly, partial suppression of gp130/Jak2/Stat3 signalling is sufficient to limit growth of Wnt-dependent intestinal tumours in models of familial and sporadic CRC through Bmi1-dependent p16 and p21 induction (Bracken et al., 2007; Fasano et al., 2007). Consistent with this, tumour burden remained unaffected in gp130/Jak2/Stat3 signalling-proficient Apc-mutant mice following genetic ablation of p16 or p21 (Cole et al.,2010).

Despite extensive efforts to therapeutically target excessive Wnt signalling, progress has proven difficult, and recent strategies focused instead on rate-limiting unmutated pathways (van Es et al., 2005; Fujishita et al., 2008). By determining the mechanism through which the gp130/Jak2/Stat3 pathway limits proliferation of normal and transformed IECs selectively in situations of excessive Wnt signalling, the inventors provide proof for the development of existing gp130/Jak2/Stat3 pathway inhibitors into therapeutic modalities for CRC and other diseases involving excessive Wnt signalling. This is surprising in light of previous reports that targeting Stat3 signalling pathways may have adverse effects on tumour progression (Musteanu et al., 2010).

### Example 2

### Functional dependence of human APC-mutant tumors cells on intact GP130/JAK/STAT3 signalling

The present inventors defined expression signatures for Wnt-signaling (Phesse et al., 2008; de Lau et al., 2011) and Stat3-signaling (Oh et al., 2009; Snyder et al., 2008) and interrogated two independent human gene expression sets (Hong et al., 2010; Skrzypczak et al., 2010). They observed a highly significant correlation between these two signatures among the cancer samples when compared to their expression in matched normal colons (FIG. 8). Collectively, these observations indicate a functional dependence of human APC-mutant tumors cells on intact GP130/JAK/STAT3 signalling.

### Example 3

### Homeostatic turnover of the intestinal epithelium in mice treated With AZD1480

Since epithelial Stat3 is essential for survival of intestinal stem cells (Matthews et al., 2011), the inventors assessed homeostatic turnover of the intestinal epithelium in mice treated for 3 weeks with AZD1480. The inventors detected no differences in the proportion of BrDU-positive proliferating intestinal epithelium cells, of differentiated mucus-producing and PAS-staining goblet cells, or of the lysozyme-positive Paneth cells, which help maintain the identity of Lgr5+ stem cells (FIG. 9b). Likewise, long-term AZD1480 administration did not affect body weight (FIG. 9c) consistent with the observation that intestinal expression of many prototypical Wnt target genes remained unaffected (FIG. 9a).

### Example 4

### Inhibition of Jak1/2 activity is sufficient to arrest the growth of Wnt-driven tumours

In light of the findings that systemic administration of a small molecule Jak1/2 inhibitor reduced intestinal tumour burden of Apc^{Min} and of Lgr5^{CreERt2};Apc^{fl/fl/} mice, the inventors confirmed that this effect is due to on target activity of these compounds by using the unrelated CYT387 inhibitor. Following administration of tamoxifen to confer cre-recombinase mediated Apc gene inactivation, and hence aberrant Wnt activation, the inventors systemically administered CYT387 for 4 weeks before analysing intestinal tumour burden. While histological assessment showed the expected large tubular adenomas in mice of the vehicle-treated control groups, the few small emerging lesion in the small and large intestines of the CYT387-treated cohort retained the morphology of the growth arrested microadenomas observed in Lgr5^{CreERt2};Apc^{fl/fl};Stat^{+/fl} (FIG. 10). These observations indicate that pharmacological inhibition of Jak1/2 activity is sufficient to arrest the growth of Wnt-driven tumours.

The present application claims priority from AU 2013901161 filed 4 Apr. 2013, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida and Allshire (2005) Trends Cell Biol, 15:251-258.
Ashton et al. (2010) Dev. Cell 19:259-269.
Barker et al. (2009) Nature 457:608-611.
Bird et al. (1988) Science, 242:423-126.
Bollrath et al. (2009) Cancer Cell 15:91-102.
Bracken et al. (2007) Genes Dev. 21:525-530.
Capdevila et al. (2001) Annu Rev Cell Dev Biol. 17:87-132.
Cole et al. (2010) EMBO Molecular Medicine 2:472-486.
Constantini et al. (2008) Cancer Biotherm Radiopharm, 23: 3-24.
Cordero (2012) EMBO J 31:3901-3917.
De Coupade et al. (2005) Biochem J, 390:407-418.
De Lau et al. (2011) Nature 476:293-297.
Deshayes et al. (2008) Adv Drug Deliv Rev, 60:537-547.
Durand et al. (2011) J Clin Endocrinol Metab 96:E1206-1211.
Ernst et al. (2001) The Journal of Experimental Medicine 194:189-203.
Ernst et al. (2008) The Journal of Clinical Investigation 118:1727-1738.
Fasano et al. (2007) Cell Stem Cell 1:87-99.
Faux et al. (2004) Journal of Cell Science 117:427-439.
Fodde & Brabletz (2007) Curr. Opin. Cell Biol. 19: 150-158.
Fujishita et al (2008) PNAS USA 105:13544-13549.
Harmsen and De Haard (2007) Appl Microbiol Biotechnol, 77:13-22.
Hazra et al. (2008) Cancer Causes Control 19:975-980.
Hedvat et al. (2009) Cancer Cell 16: 487-497.
Hong et al. (2010) Clin Exp Metastasis 27: 83-90.
Howl et al. (2007) Biochem Soc Trans 35:767-769.
Huston et al. (1988) Proc Natl Acad Sci. USA. 85:5879-5883.
Jones et al. (1986) Nature. 321:522-525.
Jin et al. (2003) Int. J. Cancer 107:696-699.
Klaus and Birchmeier (2008) Nature Reviews 8:387-398.
Koppelhus et al. (2008) Bioconj Chem, 19:1526-1534.
Lazar et al. (2008) Am. J. Path. 173:1518-1527.
Lin et al. (2011) Mol Hum Reprod 17:605-611.
Livak and Schmittgen (2001) Methods 25:402-408.
Logan and Nusse (2004) Annu Rev Cell Dev Biol. 20:781-810.
Lovig et al. (2002) Scand J Gastroent 10:1184-1193.
Matthews et al. (2011) Cell Death and Diff 18:1934-1943.
Meydan et al. (1996) Nature 379: 645-648.
Meyer-Losic et al. (2006) J Med Chem, 49:6908-6916.
Millar and Waterhouse (2005) Funct Integr Genomics, 5:129-135.
Morrison et al. (1984) Proc Natl Acad Sci USA, 81:6851-6855.
Musteanu et al. (2010) Gastroenterology 138:1003-1011.
Muyldermans (2001) J Biotechnol 74:277-302.
Noffsinger (2009) Annu Rev Pathol. 4:343-364.
Oh et al. (2009) PLoS One 4:e6911.
Ou et al. (2011) Mol Cancer Res. 9:660.
Pan et al. (2008) World J Gastroent 14:1570-1574.
Pasquinelli et al. (2005) Curr Opin Genet Develop, 15:200-205.
Perriman et al. (1992) Gene, 113:157-163.
Phesse et al. (2008) Mol. Cell Biol. 28:6094-6103.
Polakis (2000) Genes Dev. 14:1837-1851.
Sansom et al. (2004) Genes Dev. 18:1385-1390.
Sato et al. (2009) Nature 459: 262-265.
Sato et al. (2011) Nature 469:415-418.
Scheel et al. (2010) BMC Cancer 10:413.
Shippy et al. (1999) Mol Biotech, 12:117-129.
Siddiquee et al. (2007) PNAS USA 104:7391-7396.
Skrzypczak et al. (2010) PLoS One 5:e13091.
Slattery et al. (2008) Cancer Epidemiol Biomark Prev 17:978-982.
Snyder et al. (2008) J Biol Chem 283:3791-3798.
Tibary et al. (2007) Soc Reprod Fertil Suppl 64:297-313.
Traverso et al. (2002) N. Engl. J. Med. 346:311-320.
Vallania et al. (2009) PNAS USA 106:5117-5122.
van Amerongen et al. (2009) Development. 136:3205-3214.
van Es et al. (2005) Nature 435:959-963.
Varesco et al. (1993) Am. J. Hum. Genet. 52:280-285.
Yu et al (2009) Nature Reviews 9:798-809.

1. A method of treating or preventing a disease characterized by excessive Wnt signalling in a subject, the method comprising administering to the subject a compound which inhibits the activity of one or more of JAK2, JAK1 or TYK2.

2. The method of claim 1, wherein the disease is cancer.

3. The method of claim 2, wherein the cancer selected from colorectal cancer, hepatocellular cancer, medullablastoma, ovarian cancer, pancreatic cancer, gastric cancer, endometrial cancer, adrenocortical cancer, pituitary gland cancer, biliary tract cancer, kidney cancer, soft tissue cancer, intestinal cancer, breast cancer, oesophageal cancer, gliobalstoma, lung cancer, prostate cancer and thyroid cancer.

4. The method of claim 3, wherein the cancer is colorectal cancer.

5. The method according to any one of claims 1 to 4, wherein the subject has one or more mutations or epigenetic modifications in a gene encoding a protein involved in Wnt signalling, and wherein the gene is selected from APC, TCF7L2 (=TCF4), CTNNB1, WTX, AXIN1, DKK or a SFRP.

6. The method of claim 5, wherein the subject has one or more mutations or epigenetic modifications in the APC gene.

7. The method according to any one of claims 1 to 6, wherein the subject is a human.

8. The method according to any one of claims 1 to 7, wherein the compound is selected from Ruxolitinib, Baricitinib, Lestaurtinib, Pacritinib, CEP-33779, SB1578, SB1317, TG101348, TG101209, CYT387, AZD1480, AZ960, LY2784544, BMS911543, SGI-1252, MK0457, XL019, AG490, AT9283, NVP-BSK805, AC430 and GLPG0634.

9. The method of claim 8, wherein the compound is AZD1480.

10. The method according to any one of claims 1 to 7, wherein the compound is a selective inhibitor of JAK1.

11. The method of claim 10, wherein the compound is selected from GLPG0634,

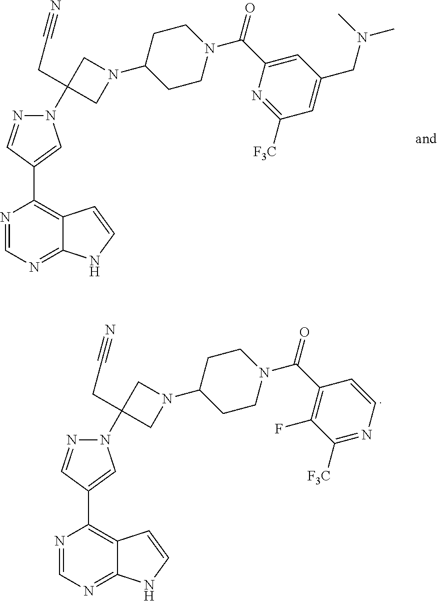 [SMILES: CN(C)Cc1cc(C(=O)N2CCC(N3CC(CC#N)(n4cc(-c5ncnc6[nH]ccc56)cn4)C3)CC2)nc(C(F)(F)F)c1 ; N#CCC1(n2cc(-c3ncnc4[nH]ccc34)cn2)CN(C2CCN(C(=O)c3ccnc(C(F)(F)F)c3F)CC2)C1]

12. The method according to any one of claims 1 to 7, wherein the compound is a selective inhibitor of JAK2.

13. The method of claim 12, wherein the compound is selected from Pacritinib, CEP-33779, SB1578, TG101348, TG101209, AZD1480, AZ960, LY2784544, BMS911543, SGI-1252, MK0457, XL019 and NVP-BSK805.

14. The method of claim 13, wherein the compound is AZD1480.

15. The method according to any one of claims 1 to 7, wherein the compound is a selective inhibitor of JAK1 and JAK2.

16. The method of claim 15, wherein the compound is selected from Ruxolitinib, Baricitinib and CYT387.

17. The method according to any one of claims 1 to 7, wherein the compound is a selective inhibitor of TYK2.

18. The method of claim 17, wherein the compound is selected from TG101348,

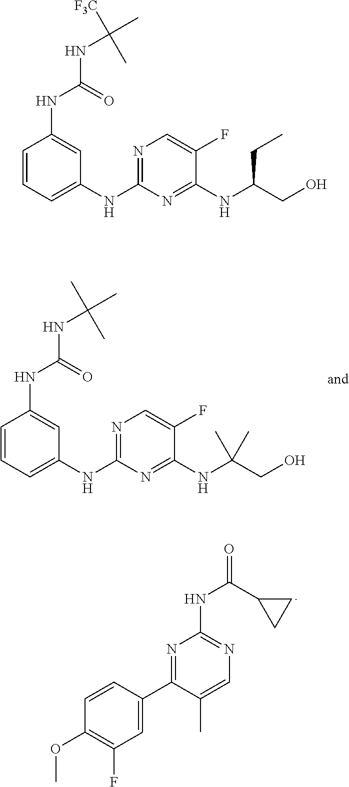 [SMILES: CC[C@@H](CO)Nc1nc(Nc2cccc(NC(=O)NC(C)(C)C(F)(F)F)c2)ncc1F ; CC(C)(C)NC(=O)Nc1cccc(Nc2ncc(F)c(NC(C)(C)CO)n2)c1 ; COc1ccc(-c2nc(NC(=O)C3CC3)ncc2C)cc1F]

19. The method according to any one of claims 1 to 7, wherein the compound is an antibody which binds one or more of JAK2, JAK1 or TYK2.

20. The method according to any one of claims 1 to 7, wherein the compound reduces transcription and/or translation of a gene encoding one or more of JAK2, JAK1 or TYK2.

21. The method of claim 20, wherein the compound is a polynucleotide.

22. The method of claim 21, wherein the polynucleotide is selected from: an antisense polynucleotide, a sense polynucleotide, a catalytic polynucleotide, a microRNA and a double stranded RNA.

23. Use of a compound which inhibits the activity of one or more of JAK2, JAK1 or TYK2 for the manufacture of a medicament for treating or preventing a disease characterized by excessive Wnt signalling in a subject.

24. Use of a compound which inhibits the activity of one or more of JAK2, JAK1 or TYK2 as a medicament for treating or preventing a disease characterized by excessive Wnt signalling in a subject.

25. A method of treating or preventing cancer in a subject, the method comprising;
i) analysing a sample from the subject to determine whether cells of the subject have excessive Wnt signalling, and
ii) if excessive Wnt signalling is detected in step i), administering to the subject a compound which inhibits the activity of one or more of JAK2, JAK1 or TYK2.

26. The method of claim 25, wherein the subject has cancer and the sample comprises cancerous cells.

27. The method of claim 25 or claim 26, wherein step i) comprises analysing DNA in the sample.

28. The method according to any one of claims 25 to 27, wherein step i) comprises analysing the sample for one or more mutations or epigenetic modifications in a gene encoding a protein involved in Wnt signalling, and wherein the gene is selected from APC, TCF7L2 (=TCF4), CTNNB1, WTX, AXIN1, DKK or a SFRP.

29. Use of a compound which inhibits the activity of one or more of JAK2, JAK1 or TYK2 for the manufacture of a medicament for treating or preventing a disease characterized by excessive Wnt signalling in a subject, wherein the subject has been identified as having excessive Wnt signalling.

30. Use of a compound which inhibits the activity of one or more of JAK2, JAK1 or TYK2 as a medicament for treating or preventing a disease characterized by excessive Wnt signalling in a subject which has been identified as having excessive Wnt signalling.

31. A method of stratifying individuals in a clinical trial of a compound which inhibits the activity of one or more of JAK2, JAK1 or TYK2, the method comprising,
i) analysing samples from the individuals to determine whether cells in the samples have excessive Wnt signalling, and,
ii) selecting individuals for the trial who have excessive Wnt signalling.


SEQUENCE LISTING (2 sequences)

SEQ ID NO: 1 - DNA, 26 bp, Artificial Sequence
  Primer
aagctcgagagggtttaagcaccttg

SEQ ID NO: 2 - DNA, 29 bp, Artificial Sequence
  Primer
aagagatctcccaaacctgcagcaactat